(19) United States
(12) Patent Application Publication
(10) Pub. No.: US 2003/0148294 A1
(43) Pub. Date: Aug. 7, 2003

(54) DNA encoding novel chemokine receptors

(76) Inventors: Janice Au-Young, Berkeley, CA (US); Olga Bandman, Mountain View, CA (US); Roger Coleman, Mountain View, CA (US); Craig G. Wilde, Sunnyvale, CA (US)

Correspondence Address: INCYTE GENOMICS, INC., PATENT DEPARTMENT, 3160 Porter Drive, Palo Alto, CA, 94304, US

(21) Appl. No.: 10/095,876

(22) Filed: Mar. 11, 2002

Related U.S. Application Data
(63) Continuation of application No. 08/638,081, filed on Apr. 26, 1996, now abandoned.

(51) Int. Cl. C12Q 1/68; C07H 21/04; C12P 21/02; C07K 14/715

(52) U.S. Cl. 435/6; 435/69.5; 435/69.1; 435/320.1; 435/325; 530/350; 536/23.5

(57) ABSTRACT

The present invention provides polynucleotides (mmlr-ccr or mphg-ccr) which encode novel chemokine receptors (MMLR-CCR OR MPHG-CCR). The present invention provides for screening methods for the detection of molecules that modulate receptor activity. The present invention also provides for antisense molecules, diagnostic molecules, genetically engineered expression vectors and host cells for the production of purified MMLR-CCR or MPHG-CCR; antibodies, agonists, antagonists and inhibitors of MMLR-CCR or MPHG-CCR; and pharmaceutical compositions and methods of treatment based on the polypeptide, its antibodies, antagonists and inhibitors. The invention further provides diagnostic and therapeutic compositions for the detection and treatment of infection, inflammation, proliferative disease, solid tumors and cardiovascular disease.

This application is a continuation application of co-pending U.S. application Ser. No. 08/638,081, entitled “DNA Encoding a Novel Chemokine Receptor,” filed Apr. 26, 1996, the disclosure of which is incorporated herein by reference.

### FIELD OF THE INVENTION

The present invention relates generally to the field of molecular biology and specifically to novel nucleotide and amino acid sequences of two chemokine receptors. The present invention further relates to the use of the novel nucleotide and amino acid sequences disclosed herein in the diagnosis and treatment of disease.

### BACKGROUND

The chemokines are small, chemotatic polypeptides, generally about 70-100 amino acids in length, 8-11 kD in molecular weight and active over a 1-100 ng/ml concentration range, that mediate the migration of leukocytes toward sites of infection. Initially, they were isolated and purified from inflamed tissues and characterized relative to their bioactivity. More recently, chemokines have been discovered through molecular cloning techniques and characterized by structural as well as functional analysis.

The chemokines are related through both primary structure and the presence of a cysteine motif which is based primarily on the spacing of the first two cysteine residues in the mature molecule. Currently the chemokines are assigned to one of two families, the C-X-C chemokines (α) and the C-C chemokines (β). Although exceptions exist, the C-X-C chemokines generally activate neutrophils and fibroblasts while the C-C chemokines act on a more diverse group of target cells which include monocytes/macrophages, basophils, eosinophils, T lymphocytes and others. The C-X-C class includes melanoma growth-stimulating activity (MGSA), interleukin-8 (IL-8) and neutrophil-activating peptide 2 (NAP-2). The C-C class includes RANTES (Regulated on Activation, Normal T Expressed and Secreted), macrophage inflammatory protein-1α and -1β (MIP-1α and -1β) and monocyte chemotactic protein-1 (MCP-1).

MCP-1 is a potent and specific monocyte agonist and chemoattractant that is produced by endothelial cells, smooth muscle cells, and macrophages in response to mediators including platelet-derived growth factor, tumor necrosis factor α, lipopolysaccharide and oxidized low density lipoproteins. MCP-1 has been implicated in mediating monocytic infiltration of tissues in inflammatory diseases, such as rheumatoid arthritis, alveolitis, atherosclerosis, and macrophage infiltration of tumors where it may be involved in the suppression of tumor growth in animal models (Charo 1994 Proc. Natl. Acad. Sci. 91: 2752-2756). Charo supra report that monocyte invasion in the artery wall is a critical event in the initiation of atherosclerosis and that MCP-1 is highly expressed in the active, macrophage-rich areas of human atherosclerotic plaques. Two other related proteins (MCP-2 and MCP-3) were purified from a human osteosarcoma cell line and have 62% and 73% amino acid identity, respectively, with MCP-1 and share its chemoattractant specificity for monocytes.

In addition to leukocyte trafficking, both C-C and C-X-C chemokines are implicated as major participants in acute as well as chronic inflammatory conditions, inhibition of hematopoiesis, modulation of angiogenesis and fibroplasia (Taub et al.1994 Ther. Immunol. 1: 229-246). Macrophage inflammatory protein 1 alpha (MIP-a) has been reported to inhibit the proliferation of hematopoietic stem cells in vitro and in vivo (Cook J. 1996 Leukoc. Biol.59:61-66) and Gewirtz et al. (1995 Blood 86: 2559-2567) report that three C-C chemokines, MIP-1a and MIP-1β and c10, specifically inhibited megakaryocyte colony formation at neutrophil activating peptide-2 (NAP-2) equivalent doses. Eaves et al. (1993 Proc. Natl. Acad. Sci USA 90: 12015-12019) suggest the administration of MIP-1a to patients with chronic myeloid leukemia (CML) for the protection of primitive normal myeloid cells.

Chemokines produce their biological effects by interacting with specific receptors on the cell surface of their target cells, i.e., immune cells. The known chemokine receptors are basic proteins that internalize after binding and are coupled to G-proteins. Additionally, they display amino acid sequences which are predicted to conform to an architecture containing seven-transmembrane spanning segments connected by a series of intra- and extracellular loops. C-X-C and CC chemokine receptors are highly specific and there is no cross-competition for binding between the two different classes of chemokines. The exception to the rule is the erythrocyte chemokine receptor that binds both C-C and C-X-C chemokines with high affinity and has been shown to be a receptor for the malarial parasite, Plasmodium vivax. There are features of the erythrocyte chemokine receptor that distinguish it from the previously characterized receptors, however. The erythrocyte chemokine receptor is an acidic protein that does not internalize after binding and is not regulated by G proteins. (Horuk 1994 Trends Pharmacol Sci 15: 159-165).

At least two C-X-C chemokine receptors, IL8_{A} and IL8_{B}, have been reported by Holmes et al (1991 Science 253: 1278-1280) and Murphy et al (1991 Science 253: 1280-1283), respectively. Three C-C receptor types, the MIP-1 α/RANTES type (CCR-1), the MCP-1 type (CCR-2) and CCR-3, (initially derived from murine eosinophils) have been reported (Post supra). Charo supra, report on the cloning of two MCP-1 receptor isoforms that are most closely related to the MIP-1 α receptor. Charo supra report on the presence of the amino acid sequence of SEQ ID NO: 5 or SEQ ID NO: 6 in the MCP-1 receptors which occurs in the third transmembrane domain and second intracellular loop and is known to interact with G-binding proteins in rhodopsin. Charo supra suggest that this domain may mediate aspects of C-protein activation common to receptors for C-C chemokines.

The discovery of new chemokine receptors associated with monocyte/macrophage chemotaxis will provide the basis for the development of new methods for screening for modulators of receptor activity and will aid in the development of diagnostic and therapeutic agents for inflammation, infection and conditions associated with abnormal proliferation of cells.

### SUMMARY

The present invention relates to a novel chemokine receptor, designated herein as MMLR-CCR. The nucleotide sequence of MMLR-CCR was found among the sequences of a cDNA library made from plastic adherent mononuclear cells collected on day two of a two-way mixed lymphocyte (MLR) culture. The present invention also relates to a novel chemokine receptor, designated herein as MPHG-CCR. The nucleotide sequence of MPHG-CCR was found among the sequences of a cDNA library made from plastic adherent (two hour culture) mononuclear cells. The present invention relates to the use of the nucleotide and amino acid sequences for the two chemokine receptors disclosed herein in the study, diagnosis and treatment of disease states in which normal leukocyte functioning is perturbed by either normal leukopoiesis or by inappropriate activation via chemokine agonists or antagonists, such as in infection, inflammation, proliferative diseases, tumorigenesis, and cardiovascular disease.

The present invention also relates to the use of MMLR-CCR and MPHG-CCR and genetically engineered host cells that express MMLR-CCR and MPHG-CCR to evaluate, screen and identify 1) the naturally occurring, biological ligands, i.e. chemokines, in appropriate cellular supernatants and 2) substances, compounds or synthetic drugs that modulate receptor activation, by modulating receptor/ligand binding, thereby modulating signal transduction events. Such genetically engineered host cells could be used, for example, to screen peptide libraries or organic molecules capable of modulating receptor/ligand binding.

The present invention is based in part on the amino acid homology that MMLR-CCR shares with the MCP-1 CCR-2 chemokine receptor, MCP-1RB, disclosed in GenBank, NCBI gi: 472557 (Charo supra) and the amino acid homology that MPHG-CCR shares with C-C chemokine receptor 3 (g881570) and MCP-1RA receptor (g472556) disclosed in Charo supra. In particular, MMLR-CCR contains the conserved amino acid sequence IFFIILLTIDRYLAVVHAVFAL (K/R) ARTVFGV, found in the third transmembrane domain and second intracellular loop of chemokine receptors which in the corresponding region of rhodopsin is known to participate in G-protein binding, suggesting that this domain may mediate aspects of G-protein activation common to receptors for C-C chemokines. There is one conserved amino acid substitution in the G-protein binding domain at position 121 in MMLR-CCR which is valine instead of isoleucine as is reported by Charo supra.

The present invention is therefore based on the discovery of two novel C-C chemokine receptors, MMLR-CCR and MPHG-CCR, that may be associated with monocyte/macrophage infiltration and chemotaxis and hematopoiesis. MMLR-CCR and MPHG-CCR and nucleotide sequences that encode them and oligonucleotides, peptide nucleic acid (PNA), fragments, portions or antisense molecules thereof, provide the basis for diagnostic methods for the detection and/or quantitation of a MMLR-CCR associated with infection, inflammation, proliferative diseases, tumorigenesis, and cardiovascular disease, including such diseases as rheumatoid arthritis; alveolitis; atherosclerosis; chronic granulomatous disease (characterized by an extensive inflammatory reaction); asthma; autoimmune diseases, such as myasthenia gravis, diabetes and inflammatory bowel disease; toxic shock syndrome; septic shock; Chediak-Higashi syndrome (characterized by decreased microbicidal killing); conditions associated with abnormal proliferation of cells; and solid tumors. For example, the mmlr-ccr nucleotide sequence disclosed herein, which encodes MMLR-CCR, or fragments thereof, may be used in hybridization assays of biopsied cells or tissues or bodily fluids to detect mmlr-ccr nucleic acid which may be associated with such disease states.

An abnormal level of mmlr-ccr or mphg-ccr nucleotide sequences or an abnormal transcript size in a biological sample may be characteristic of a regulatory state in which the receptors are over-expressed or under-expressed. Nucleotide sequences encoding MMLR-CCR or MPHG-CCR provide the basis for probes which can be used diagnostically to detect chromosomal aberrations such as deletions, mutations or chromosomal translocations in the gene encoding the chemokine receptors. Gene expression may be altered in such disease states or there may be a chromosomal aberration present in the region of the gene encoding the receptor.

The present invention also relates, in part, to expression vectors and genetically engineered host cells comprising nucleotide sequences encoding MMLR-CCR or MPHG-CCR for in vitro or in vivo production of the nucleotide sequences.

Additionally, the present invention relates to the use of a MMLR-CCR or MPHG-CCR polypeptide, or fragment or variant thereof, to produce antibodies and to screen for antagonists or inhibitors of the chemokine receptors which can be used diagnostically to detect and quantitate protein levels in disease states.

Peptides or small molecules capable of modulating receptor levels or activity will provide the basis for pharmaceutical compositions for the treatment of disease states associated with receptor activtation, such as inflammation. Chemokine receptor antisense molecules may be used to down-regulate the presence of the receptor on the cell surface in conditions where it is preferable to decrease receptor signalling. Alternatively, antagnoists of the chemokine receptor/ligand interaction may be used to block receptor/ligand binding in diseases or conditions where it is preferable to block or reduce receptor activation, i.e., signal transduction events which lead to infiltration/chemotaxis, and a variety of other second messenger mediated cellular events. Alternatively, mmlr-ccr or mphg-ccr sense molecules may be used to up-regulate the presence of the receptor and MMLR-CCR or MPHG-CCR agonists may be used to enhance receptor activation thereby enhancing signal transduction events in conditions where it is desirable to increase chemotaxis and other second messenger mediated cellular events. Such molecules capable of modulating receptor activity can be administered alone or in combination with other therapeutics for the treatment of diseases.

The invention further provides diagnostic assays and kits for the detection of MMLR-CCR or MPHG-CCR in cells and tissues comprising one of the purified receptors which may be used as a positive control, and anti-receptor antibodies. Such antibodies may be used in solution-based, membrane-based, or tissue-based technologies to detect any disease state or condition related to the expression of protein or expression of deletions or variants thereof.

### DESCRIPTION OF THE FIGURES

FIGS. 1A-1B display the polynucleotide (SEQ ID NO: 1)and deduced amino acid (SEQ ID NO: 2) sequence for MMLR-CCR. The conserved sequence IFFIILLTIDRYLAVVHAVFAL (K/R) ARTVFGV begins at amino acid residue 107.

FIG. 2 displays the amino acid alignment of MMLR-CCR with the MCP-1 receptor (Charo supra). Sequences shown in this FIGURE and FIG. 7 were produced using the multisequence alignment program of DNASTAR software (DNASTAR Inc, Madison Wis.).

FIG. 3 displays an analysis of the hydrophobicity characteristics of MMLR-CCR based on the predicted amino acid sequence.

FIG. 4 displays the isoelectric point for MMLR-CCR as determined by MacDNAsis. The results indicate that the isoelectric point [pI] for MMLR-CCR is 9.69.

FIG. 5 displays the results of a Northern blot analysis using INCYTE clone 478861 which encodes MMLR-CCR, as a probe.

FIGS. 6A-6B display the polynucleotide (SEQ ID NO: 3) and deduced amino acid (SEQ ID NO: 4) sequence for MPHG-CCR.

FIG. 7 displays the amino acid alignment of MPHG-CCR with the C-C chemokine receptor (GI 881570).

FIG. 8 displays an analysis of the hydrophobicity characteristics of MPHG-CCR based on the predicted amino acid sequence.

FIG. 9 displays the isoelectric point for MPHG-CCR as determined by MacDNAsis. The results indicate that the isoelectric point [pI] for MPHG-CCR is 7.63.

### DETAILED DESCRIPTION OF THE INVENTION

The present invention relates to a novel chemokine receptor, designated herein as MMLR-CCR, the nucleotide sequence of which was initially found among the sequences of a cDNA library made from mononuclear cells collected on day two of a mixed lymphocyte (MLR) culture. The present invention also relates to a novel chemokine receptor, designated herein as MPHG-CCR, the nucleotide sequence of which was initially found among the sequences of a cDNA library made from mononuclear cells. The present invention relates to the use of the nucleotide and amino acid sequences disclosed herein in the study, diagnosis and treatment of disease states in which normal leukocyte functioning is perturbed by either by normal leukopoiesis or by inappropriate activation via chemokine agonists or antagonists, such as in infection, inflammation, proliferative diseases, tumorigenesis, and cardiovascular disease.

The present invention also relates to the use of MMLR-CCR and MPHG-CCR and genetically engineered host cells that express MMLR-CCR and MPHG-CCR to evaluate, screen and identify 1) the naturally occurring, biological ligands, i.e. chemokines, in appropriate cellular supernatants and 2) substances, compounds or synthetic drugs that modulate receptor activation, by modulating receptor/ligand binding, thereby modulating signal transduction events.

The present invention is based in part on the presence of nucleotide sequences encoding MMLR-CCR in random samples of 5654 usable sequences in a cDNA library made from monocytes of a 48 hour treated mixed lymphocyte culture, i.e. cells associated with inflammation and immunomodulation. Nucleotide sequences for MMLR-CCR are absent in a random sample of 7749 usable sequences in a cDNA library made from monocytes not subjected to a mixed lymphocyte reaction. The present invention is also based in part on the presence of nucleotide sequences encoding MPHG-CCR in random samples of 7749 usable sequences in a cDNA library made from monocytes not subjected to a mixed lymphocyte reaction. As used herein the term “usuable sequences” refers to the total number of clones in a library after the removal of vector, nucleotide repeats, contamination, and mitochondrial DNA. The present invention is further based in part on the amino acid homology that MMLR-CCR shares with the known MCP-1 chemokine receptor, MCP-1RB (GenBank accession number gi: 472557), and the presence of the conserved amino acid motif associated with G protein binding and the amino acid homology that MPHG-CCR shares with C-C chemokine receptor 3 and MCP-1RA.

The present invention therefore is based upon the identification of novel chemokine receptors, MMLR-CCR and MPHG-CCR, that are associated with disease states in which normal leukocyte functioning is perturbed by either normal leukopoiesis or by inappropriate activation by chemokine agonists or antagonists, such as in infection, inflammation, proliferative diseases, tumorigenesis and cardiovascular disease, including rheumatoid arthritis; alveolitis; atherosclerosis; chronic granulomatous disease (characterized by an extensive inflammatory reaction); asthma; autoimmune diseases, such as, myasthenia gravis, diabetes and inflammatory bowel disease; toxic shock syndrome; septic shock; Chediak-Higashi syndrome (characterized by decreased microbicidal killing); and conditions associated with abnormal proliferation of cells, such as tumorigenesis.

“Nucleic acid sequence” as used herein refers to an oligonucleotide, nucleotide or polynucleotide sequence, and fragments or portions thereof, and to DNA or RNA of genomic or synthetic origin which may be double-stranded or single-stranded, whether representing the sense or antisense strand. As used herein “amino acid sequence” refers to peptide or protein sequences or portions thereof. As used herein, lower case “mmlr-ccr” or “mphg-ccr” refers to a nucleic acid sequence whereas upper case “MMLR-CCR” and “MPHG-CCR” refers to a protein sequence. As used herein, peptide nucleic acid (PNA) refers to a class of informational molecules that have a neutral “peptide like” backbone with nucleobases that allow molecules to hybridize to complementary DNA or RNA with higher affinity and specificity than corresponding oligonucleotides (PerSeptive Biosystems 1-800-899-5858).

As used herein, MMLR-CCR and MPHG-CCR encompasses MMLR-CCR and MPHR-CCR, respectively, from any mammalian species, including bovine, ovine, murine, porcine, equine and preferably human sources, in naturally occurring or in variant form, or from any source, whether natural, synthetic, semi-synthetic or recombinant.

As used herein, “naturally occurring” refers to a MMLR-CCR or MPHG-CCR with an amino acid sequence found in nature, and “biologically active” refers to MMLR-CCR or MPHG-CCR having structural, regulatory or biochemical functions of the naturally occurring receptors. As used herein, “immunological activity” is defined as the capability of the natural, recombinant or synthetic MMLR-CCR or MPHG-CCR or any oligopeptide thereof, to induce a specific immune response in appropriate animals or cells and to bind with specific antibodies.

The term “derivative” as used herein refers to the chemical modification of MMLR-CCR or MPHG-CCR. Illustrative of such modifications is replacement of hydrogen by an alkyl, acyl, or amino group. A chemokine receptor derivative retains essential biological characteristics of the naturally occurring chemokine receptor.

As used herein, the term “purified” refers to molecules, either nucleic or amino acid sequences, that are removed from their natural environment and isolated or separated from at least one other component with which they are naturally associated.

The Coding Sequences

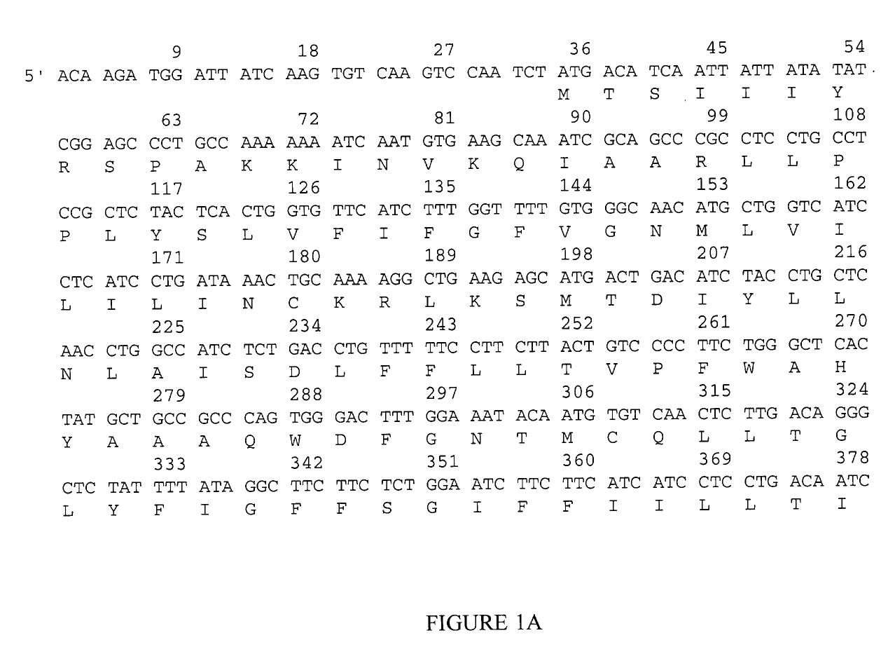

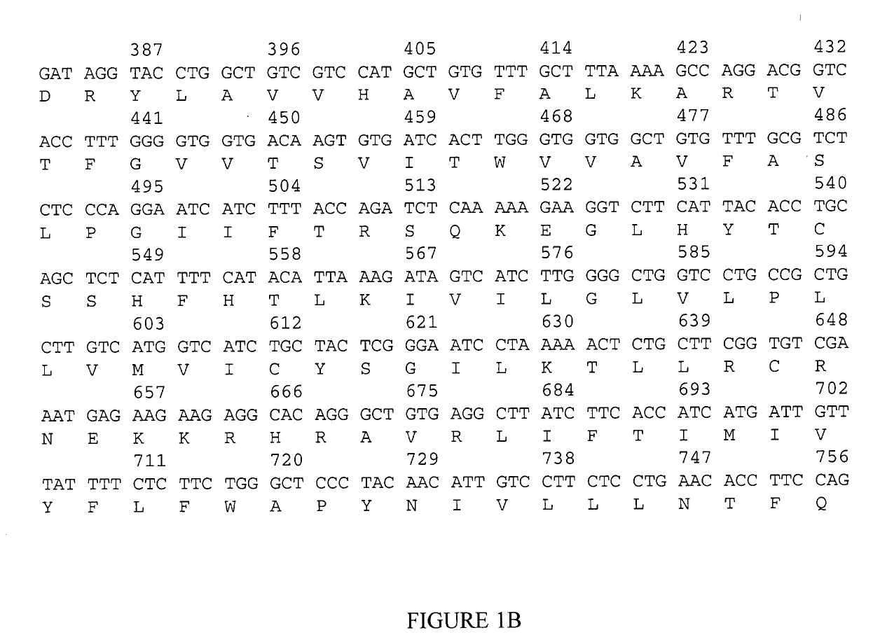

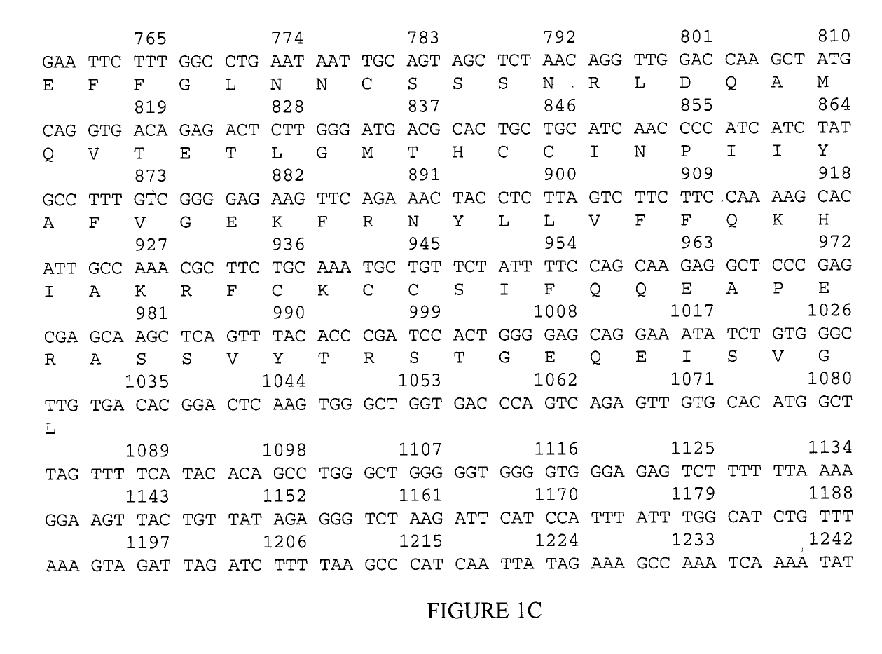

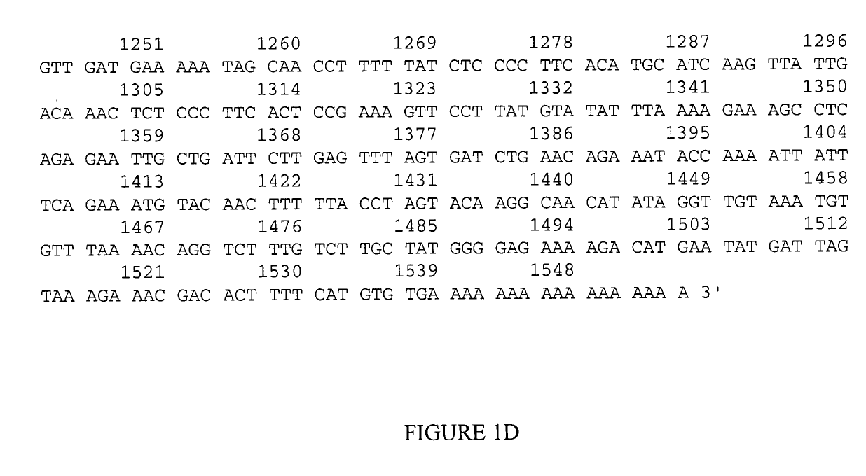

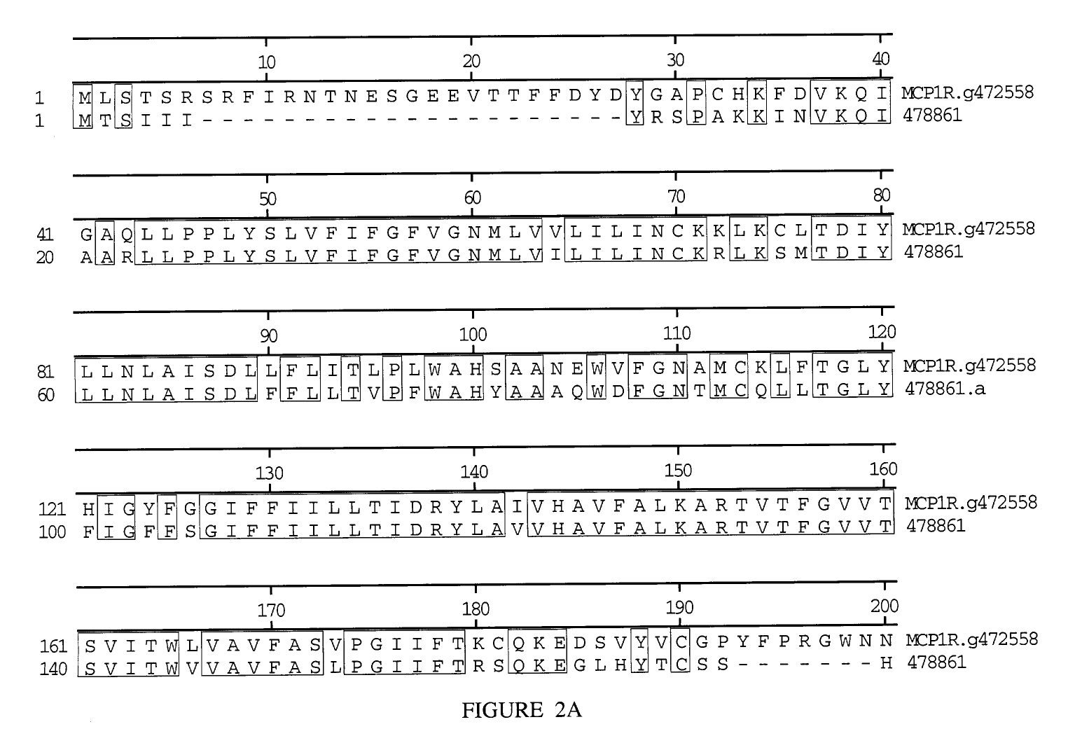

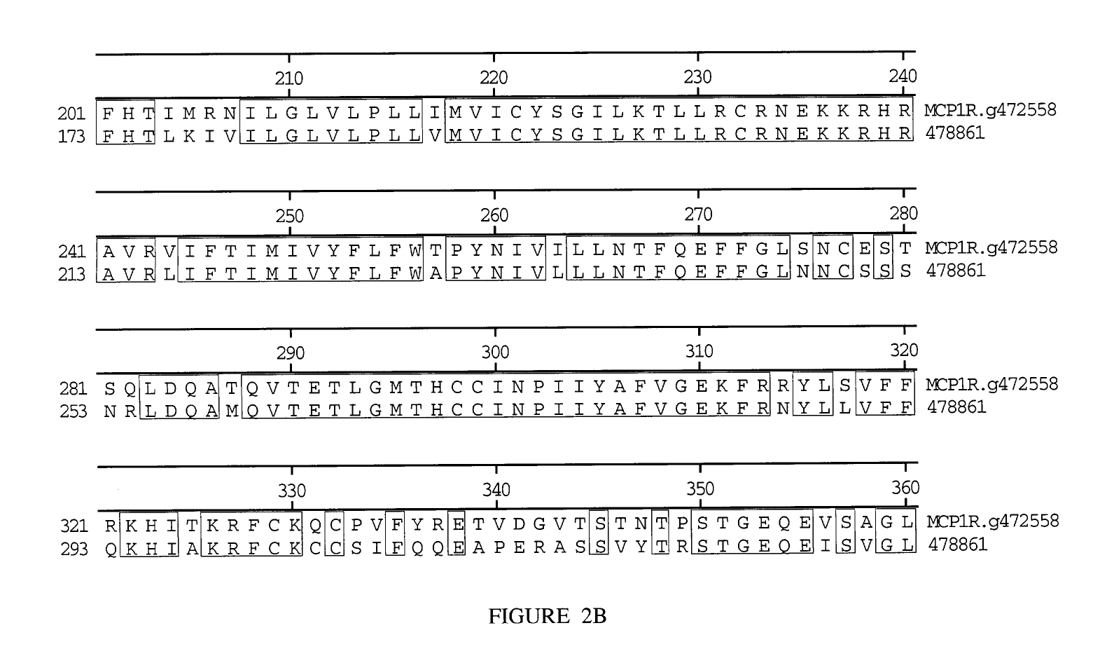

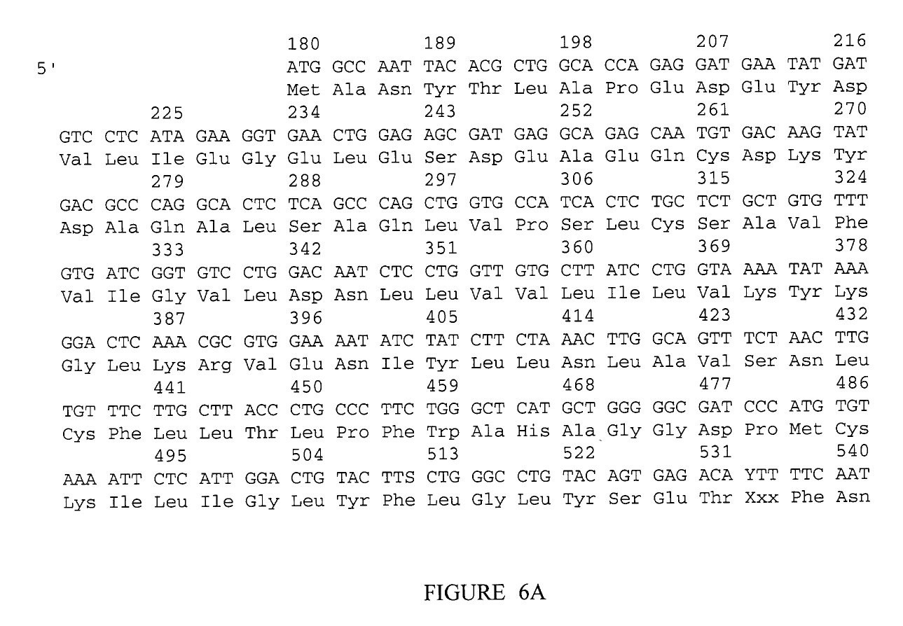

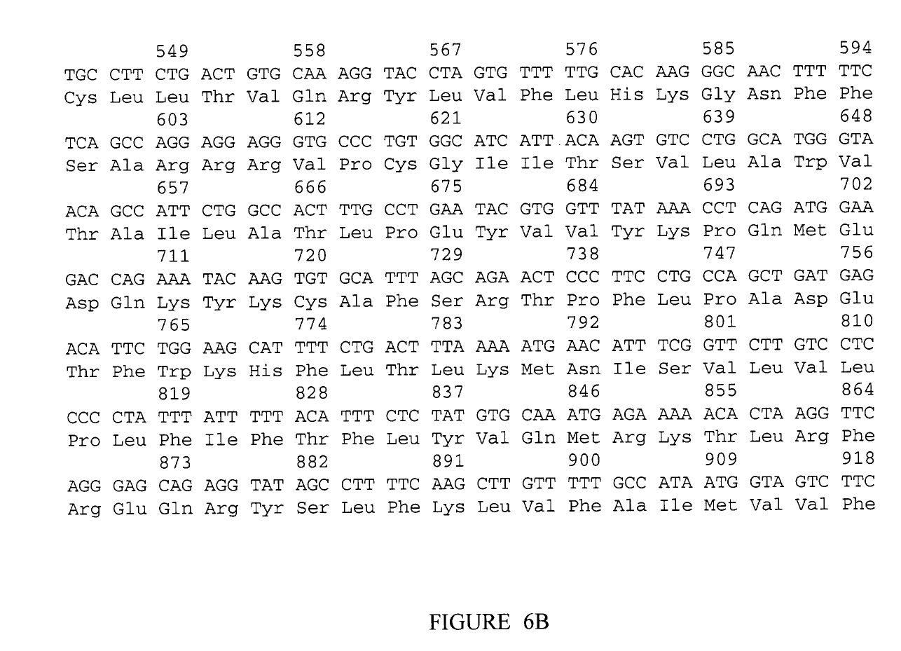

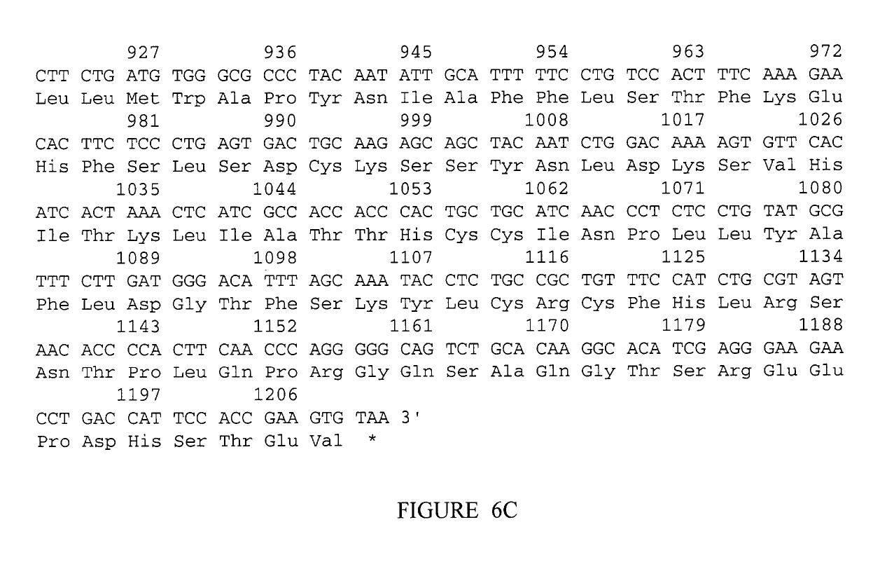

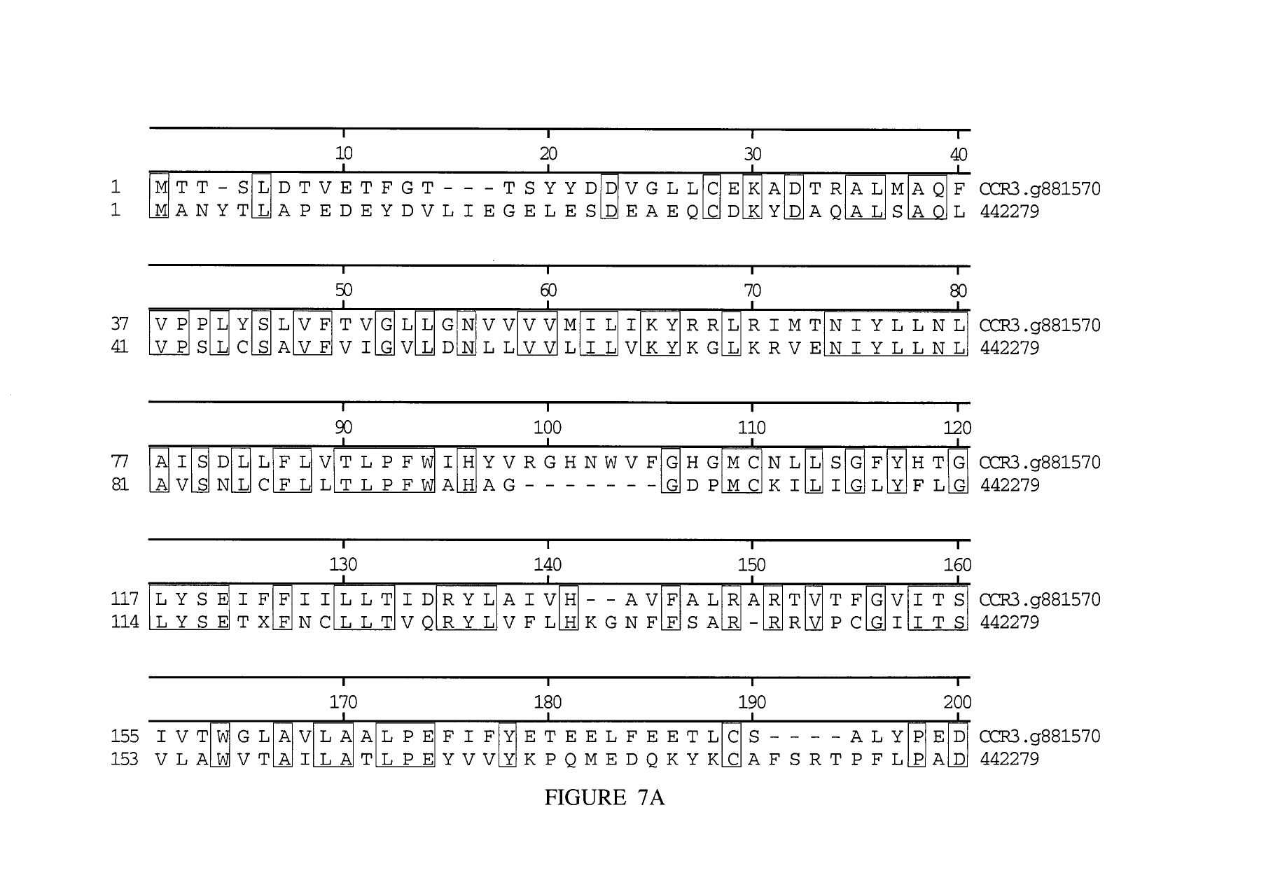

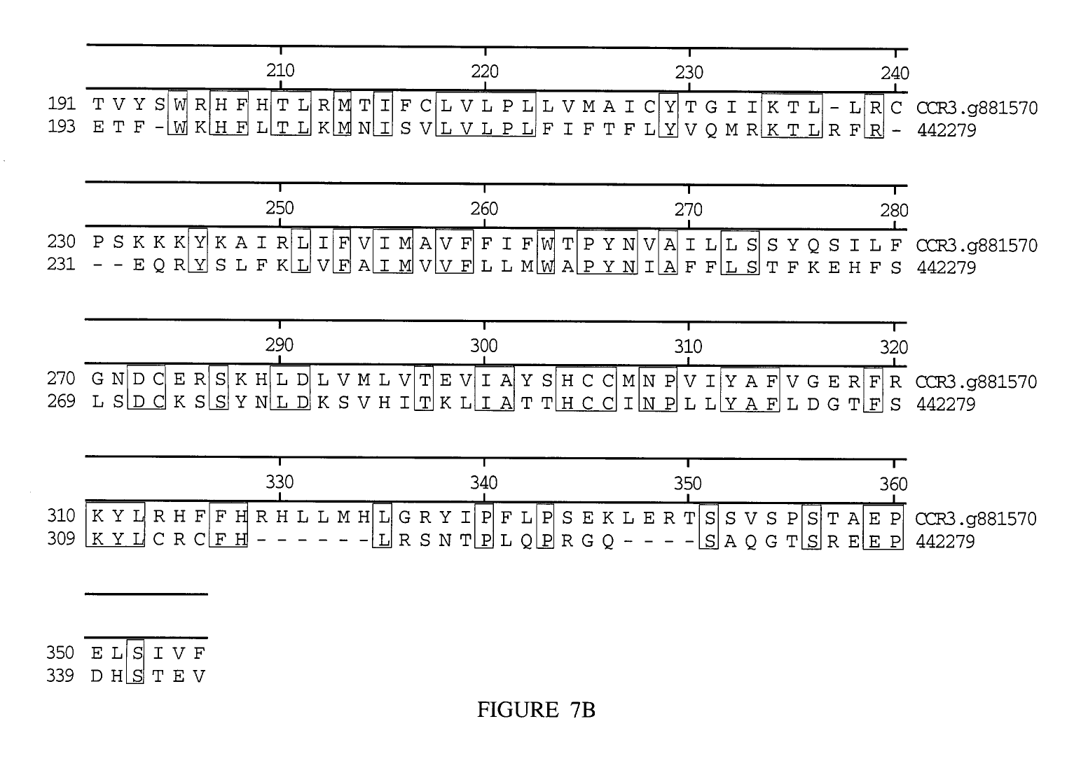

The nucleotide sequences of human mmlr-ccr (SEQ ID NO: 1) and human mphg-ccr are shown in FIGS. 1 and 6, respectively. A partial coding region for MMLR-CCR was identified within a cDNA library made from MMLR cells where it was found one time in 5654 usable sequences. A partial coding region for MPHG-CCR was identified within a cDNA library made from macrophages where it was found one time in 7749 usable sequences. A BLAST search (Basic Local Alignment Search Tool; Altschul SF (1993) J. Mol. Evol. 36: 290-300; Altschul SF et al (1990) J. Mol. Biol. 215: 403-410) comparing the human MCP-1 receptor (NCBI GI 472557) against the cDNAs of the MMLR library (INCYTE library MMLR2DT01) identified Incyte Clone 478861 (MMLR-CCR) and 442279 (MPHG-CCR). The 5′ nucleotide region of MMLR-CCR and MPHG-CCR was obtained through PCR extension and sequenced.

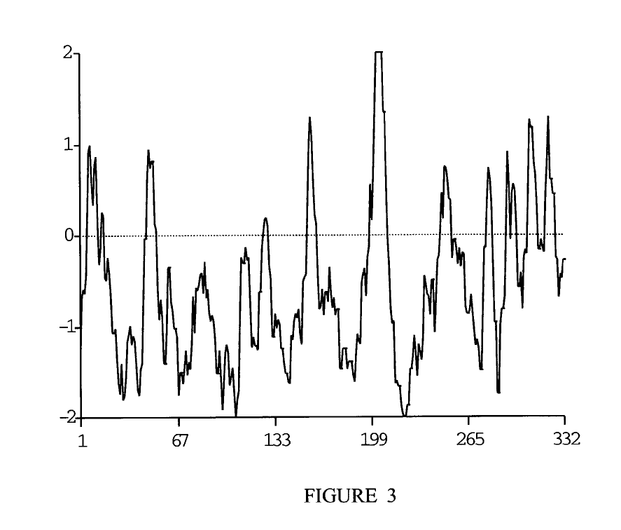

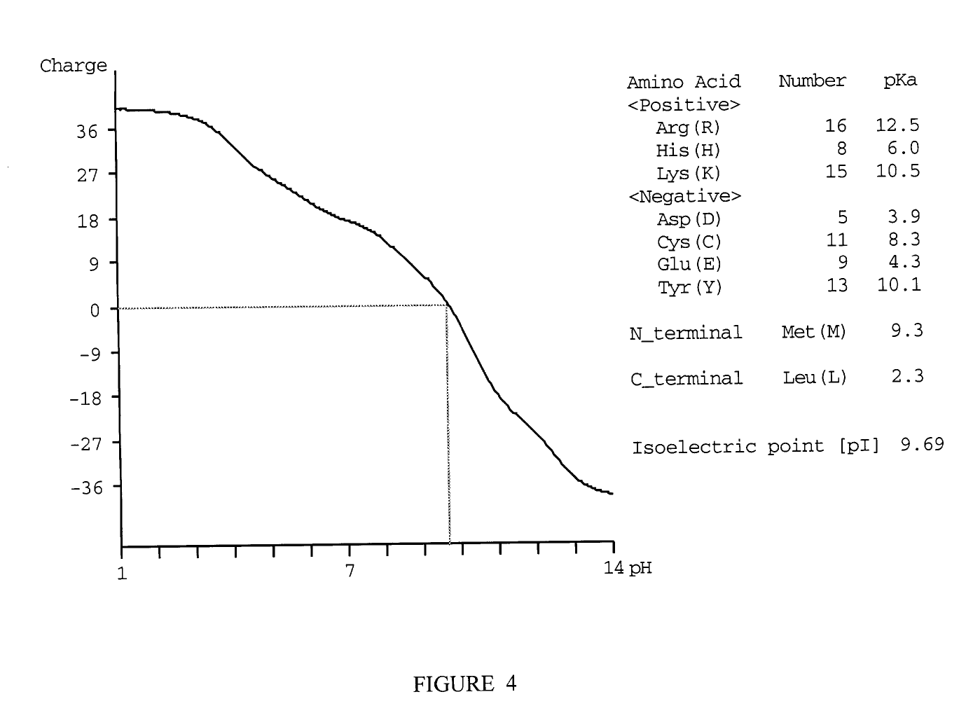

The nucleotide sequence of SEQ ID NO: 1 encodes a MMLR-CCR amino acid sequence (SEQ ID NO: 2) having 332 amino acid residues with the domain, IFFIILLTIDRYLAVVHAVFAL(K/R)ARTVFGV occurring at residue positions 107-128, inclusive, of SEQ ID NO: 2. As illustrated in FIG. 3, MMLR-CCR contains seven-transmembrane spanning segments connected by a series of intracellular and extracellular loops. The nucleotide sequence of SEQ ID NO: 3 encodes a MPHG-CCR amino acid sequence (SEQ ID NO: 4) having 344 amino acid residues.

Methods for DNA sequencing are well known in the art and employ such enzymes as the Klenow fragment of DNA polymerase I, Sequenase® (US Biochemical Corp, Cleveland Ohio)), Taq polymerase (Perkin Elmer, Norwalk Conn.), thermostable T7 polymerase (Amersham, Chicago Ill.), or combinations of recombinant polymerases and proofreading exonucleases such as the ELONGASE Amplification System marketed by Gibco BRL (Gaithersburg Md.). Methods to extend the DNA from an oligonucleotide primer annealed to the DNA template of interest have been developed for both single-stranded and double-stranded templates. Chain termination reaction products were separated using electrophoresis and detected via their incorporated, labeled precursors. Recent improvements in mechanized reaction preparation, sequencing and analysis have permitted expansion in the number of sequences that can be determined per day. Preferably, the process is automated with machines such as the Hamilton Micro Lab 2200 (Hamilton, Reno Nev.), Peltier Thermal Cycler (PTC200; M J Research, Watertown Mass.) and the ABI Catalyst 800 and 377 and 373 DNA sequencers (Perkin Elmer, Norwalk Conn.).

The quality of any particular cDNA library in which polynucleotides encoding the chemokine receptors are found may be determined by performing a pilot scale analysis of the cDNAs and checking for percentages of clones containing vector, lambda or E. coli DNA, mitochondrial or repetitive DNA, and clones with exact or homologous matches to public databases.

Extending Receptor Polynucleotide Sequences

The polynucleotide sequence encoding MMLR-CCR or MPHG-CCR may be extended utilizing the nucleotide sequences from SEQ ID NO: 1 or SEQ ID NO: 3, respectively, in various methods known in the art to detect upstream sequences such as promoters and regulatory elements. Gobinda et al (1993; PCR Methods Applic 2: 318-22) disclose “restriction-site polymerase chain reaction (PCR)” as a direct method which uses universal primers to retrieve unknown sequence adjacent to a known locus. First, genomic DNA is amplified in the presence of primer to a linker sequence and a primer specific to the known region. The amplified sequences are subjected to a second round of PCR with the same linker primer and another specific primer internal to the first one. Products of each round of PCR are transcribed with an appropriate RNA polymerase and sequenced using reverse transcriptase.

Inverse PCR can be used to amplify or extend sequences using divergent primers based on a known region (Triglia T et al(1988) Nucleic Acids Res 16: 8186). The primers may be designed using Oligo 4.0 (National Biosciences Inc, Plymouth Minn.), or another appropriate program, to be 22-30 nucleotides in length, to have a GC content of 50% or more, and to anneal to the target sequence at temperatures about 68°-72° C. The method uses several restriction enzymes to generate a suitable fragment in the known region of a gene. The fragment is then circularized by intramolecular ligation and used as a PCR template.

Capture PCR (Lagerstrom M et al (1991) PCR Methods Applic 1: 111-19) is a method for PCR amplification of DNA fragments adjacent to a known sequence in human and yeast artificial chromosome (YAC) DNA. Capture PCR also requires multiple restriction enzyme digestions and ligations to place an engineered double-stranded sequence into an unknown portion of the DNA molecule before PCR.

Parker J D et al (1991; Nucleic Acids Res 19: 3055-60 ), teach walking PCR, a method which permits retrieval of unknown sequence. PromoterFinder™ is a new kit available from Clontech (Palo Alto Calif.) which uses PCR, nested primers and special libraries to “walk in” genomic DNA. This process avoids the need to screen libraries and is useful in finding intron/exon junctions.

Another PCR method, “Improved Method for Obtaining Full Length cDNA Sequences” by Guegler et al, patent application Ser. No 08/487,112, filed Jun. 7, 1995 and hereby incorporated by reference, employs XL-PCR™ enzymes (Perkin-Elmer, Foster City Calif.) to amplify and/or extend nucleotide sequences.

Preferred libraries for screening for full length cDNAs are ones that have been size-selected to include larger cDNAs. Also, random primed libraries are preferred in that they will contain more sequences which contain the 5′ and upstream regions of genes. A randomly primed library may be particularly useful if an oligo d(T) library does not yield a full-length cDNA. Genomic libraries are useful for obtaining introns and extending 5′ sequence.

A new method for analyzing either the size or confirming the nucleotide sequence of sequencing or PCR products is capillary electrophoresis. Systems for rapid sequencing are available from Perkin Elmer, Beckman Instruments (Fullerton Calif.), and other companies. Capillary sequencing employs flowable polymers for electrophoretic separation, four different fluorescent dyes (one for each nucleotide) which are laser activated, and detection of the emitted wavelengths by a charge coupled devise camera. Output/light intensity is converted to electrical signal using appropriate software (eg. Genotyper™ and Sequence Navigator™ programs from Perkin Elmer) and the entire process from loading of samples to computer analysis and electronic data display is computer controlled. Capillary electrophoresis is particularly suited to the sequencing of small pieces of DNA which might be present in limited amounts in a particular sample. The reproducible sequencing of up to 350 bp of M13 phage DNA in 30 min has been reported (Ruiz-Martinez M C et al (1993) Anal Chem 65: 2851-8).

Expression Systems

In accordance with the present invention, mmlr-ccr or mphg-ccr polynucleotide sequences which encode MMLR-CCR or MPHG-CCR, fragments of the polypeptide, fusion proteins or functional equivalents thereof, may be used to generate recombinant DNA molecules that direct the expression of MMLR-CCR or MPHG-CCR in appropriate host cells. Due to the inherent degeneracy of the genetic code, other DNA sequences which encode substantially the same or a functionally equivalent amino acid sequence, may be used to clone and express MMLR-CCR or MPHG-CCR. As will be understood by those of skill in the art, it may be advantageous to produce chemokine receptor-encoding nucleotide sequences possessing non-naturally occurring codons. Codons preferred by a particular prokaryotic or eukaryotic host (Murray E et al (1989) Nuc Acids Res 17: 477-508) can be selected, for example, to increase the rate of expression or to produce recombinant RNA transcripts having desirable properties, such as a longer half-life, than transcripts produced from naturally occurring sequence.

Also included within the scope of the present invention are polynucleotide sequences that are capable of hybridizing to the nucleotide sequence of FIG. 1 or FIG. 6 under conditions of intermediate to maximal stringency. Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.) incorporated herein by reference, and confer a defined “stringency” as explained below.

“Maximum stringency” typically occurs at about Tm−5° C. (5° C. below the Tm of the probe); “high stringency” at about 5° C. to 10° C. below Tm; “intermediate stringency” at about 10° C. to 20° C. below Tm; and “low stringency” at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences. The term “hybridization” as used herein shall include “the process by which a strand of nucleic acid joins with a complementary strand through base pairing” (Coombs J (1994) Dictionary of Biotechnology, Stockton Press, New York N.Y.) as well as the process of amplification has carried out in polymerase chain reaction technologies as described in Dieffenbach C W and G S Dveksler (1995, PCR Primer, a Laboratory Manual, Cold Spring Harbor Press, Plainview N.Y.) and incorporated herein by reference.

As used herein a “deletion” is defined as a change in either nucleotide or amino acid sequence in which one or more nucleotides or amino acid residues, respectively, are absent.

As used herein an “insertion” or “addition” is that change in a nucleotide or amino acid sequence which has resulted in the addition of one or more nucleotides or amino acid residues, respectively, as compared to the naturally occurring MMLR-CCR or MPHG-CCR.

As used herein “substitution” results from the replacement of one or more nucleotides or amino acids by different nucleotides or amino acids, respectively.

Altered MMLR-CCR or MPHG-CCR polynucleotide sequences which may be used in accordance with the invention include deletions, insertions or substitutions of different nucleotide residues resulting in a polynucleotide that encodes the same or a functionally equivalent MMLR-CCR or MPHG-CCR. The protein may also show deletions, insertions or substitutions of amino acid residues which produce a silent change and result in a functionally equivalent receptors. Deliberate amino acid substitutions may be made on the basis of similarity in polarity, charge, solubility, hydrophobicity, hydrophilicity, and/or the amphipathic nature of the residues as long as the biological activity of the receptor is retained. For example, negatively charged amino acids include aspartic acid and glutamic acid; positively charged amino acids include lysine and arginine; and amino acids with uncharged polar head groups having similar hydrophilicity values include leucine, isoleucine, valine; glycine, alanine; asparagine, glutamine; serine, threonine phenylalanine, and tyrosine.

Included within the scope of the present invention are alleles of MMLR-CCR or MPHG-CCR. As used herein, an “allele” or “allelic sequence” is an alternative form of MMLR-CCR or MPHG-CCR. Alleles result from a mutation, ie, a change in the nucleic acid sequence, and generally produce altered mRNAs or polypeptides whose structure or function may or may not be altered. Any given gene may have none, one or many allelic forms. Common mutational changes which give rise to alleles are generally ascribed to deletions, additions or substitutions of amino acids. Each of these types of changes may occur alone, or in combination with the others, one or more times in a given sequence.

The nucleotide sequences of the present invention may be engineered in order to alter a MMLR-CCR or MPHG-CCR coding sequence for a variety of reasons, including but not limited to, alterations which modify the cloning, processing and/or expression of the gene product. For example, mutations may be introduced using techniques which are well known in the art, eg, site-directed mutagenesis to insert new restriction sites, to alter glycosylation patterns, to change codon preference, etc.

In another embodiment of the invention, a MMLR-CCR or MPHG-CCR natural, modified or recombinant sequence may be ligated to a heterologous sequence to encode a fusion protein. For example, for screening of peptide libraries for inhibitors of MMLR-CCR or MPHG-CCR activity, it may be useful to encode a chimeric chemokine receptor protein expressing a heterologous epitope that is recognized by a commercially available antibody. A fusion protein may also be engineered to contain a cleavage site located between a chemokine receptor sequence and the heterologous protein sequence, so that the MMLR-CCR or MPHG-CCR may be cleaved and purified away from the heterologous moiety.

In an alternate embodiment of the invention, the coding sequence of MMLR-CCR or MPHG-CCR could be synthesized, in whole or in part, using chemical methods well known in the art (see Caruthers M H et al (1980) Nuc Acids Res Symp Ser 215-23, Horn T et al(1980) Nuc Acids Res Symp Ser 225-32, etc). Alternatively, the protein itself could be produced using chemical methods to synthesize a MMLR-CCR or MPHG-CCR amino acid sequence, in whole or in part. For example, peptides can be synthesized by solid phase techniques, cleaved from the resin, and purified by preparative high performance liquid chromatography (eg, Creighton (1983) Proteins Structures And Molecular Principles, W H Freeman and Co, New York N.Y.). The composition of the synthetic peptides may be confirmed by amino acid analysis or sequencing (eg, the Edman degradation procedure; Creighton, supra)

Direct peptide synthesis can be performed using various solid-phase techniques (Roberge J Y et al (1995) Science 269: 202-204) and automated synthesis may be achieved, for example, using the ABI 431A Peptide Synthesizer (Perkin Elmer) in accordance with the instructions provided by the manufacturer. Additionally, the amino acid sequence of MMLR-CCR or MPHG-CCR, or any part thereof, may be altered during direct synthesis and/or combined using chemical methods with sequence from other a subunits, or any part thereof, to produce a variant polypeptide.

Identification of Transformants

Although the presence/absence of marker gene expression suggests that the gene of interest is also present, its presence and expression should be confirmed. For example, if the MMLR-CCR or MPHG-CCR is inserted within a marker gene sequence, recombinant cells containing MMLR-CCR or MPHG-CCR can be identified by the absence of marker gene function. Alternatively, a marker gene can be placed in tandem with a receptor sequence under the control of a single promoter. Expression of the marker gene in response to induction or selection usually indicates expression of the receptor as well.

Alternatively, host cells which contain the coding sequence for MMLR-CCR or MPHG-CCR and express MMLR-CCR or MPHG-CCR may be identified by a variety of procedures known to those of skill in the art. These procedures include, but are not limited to, DNA-DNA or DNA-RNA hybridization and protein bioassay or immunoassay techniques which include membrane-based, solution-based, or chip-based technologies for the detection and/or quantification of the nucleic acid or protein.

The presence of the MMLR-CCR or MPHG-CCR polynucleotide sequence can he detected by DNA-DNA or DNA-RNA hybridization or amplification using probes, portions or fragments disclosed in SEQ ID NO: 1 or SEQ ID NO: 3, respectively. Nucleic acid amplification based assays involve the use of oligonucleotides or oligomers based on SEQ ID NO: 1 or SEQ ID NO: 3 to detect transformants containing MMLR-CCR or MPHG-CCR, respectively, DNA or RNA. As used herein “oligonucleotides” or “oligomers”, refer to a nucleic acid sequence of at least about 10 nucleotides and as many as about 60 nucleotides, preferably about 15 to 30 nucleotides, and more preferably about 20-25 nucleotides which can be used as a probe or amplimer. Preferably, oligonucleotides are derived from the 3′ region of the MMLR-CCR or MPHG-CCR nucleotide sequence shown in FIG. 1.

A variety of protocols for detecting and measuring the expression of the chemokine receptor polypeptides, using either polyclonal or monoclonal antibodies specific for the protein are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes on the receptor polypeptides is preferred, but a competitive binding assay may be employed. These and other assays are described, among other places, in Hampton R et al (1990, Serological Methods, a Laboratory Manual, APS Press, St Paul Minn.) and Maddox D E et al (1983, J Exp Med 158: 1211).

A wide variety of labels and conjugation techniques are known by those skilled in the art and can be used in various nucleic and amino acid assays. Means for producing labeled hybridization or PCR probes for detecting MMLR-CCR or MPHG-CCR polynucleotide sequences include oligolabeling, nick translation, end-labeling or PCR amplification using a labeled nucleotide. Alternatively, the MMLR-CCR or MPHG-CCR sequence, or any portion of it, may be cloned into a vector for the production of an mRNA probe. Such vectors are known in the art, are commercially available, and may be used to synthesize RNA probes in vitro by addition of an appropriate RNA polymerase such as T7, T3 or SP6 and labeled nucleotides.

A number of companies such as Pharmacia Biotech (Piscataway N.J.), Promega (Madison Wis.), and US Biochemical Corp (Cleveland Ohio) supply commercial kits and protocols for these procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. Nos. 3,817,837; 3,850,752; 3,939,350; 3,996,345; 4,277,437; 4,275,149 and 4,366,241. Also, recombinant immunoglobulins may be produced as shown in U.S. Pat. No. 4,816,567 and incorporated herein by reference.

Purification of Receptors

Host cells transformed with a MMLR-CCR or MPHG-CCR nucleotide sequences may be cultured under conditions suitable for the expression and recovery of the encoded protein from cell culture. The protein produced by a recombinant cell may be secreted or may be contained intracellularly depending on the sequence and/or the vector used. As will be understood by those of skill in the art, expression vectors containing MMLR-CCR or MPHG-CCR can be designed with signal sequences which directs secretion of MMLR-CCR or MPHG-CCR through a particular prokaryotic or eukaryotic cell membrane. Other recombinant constructions may join MMLR-CCR or MPHG-CCR to nucleotide sequence encoding a polypeptide domain which will facilitate purification of soluble proteins (Kroll D J et al (1993) DNA Cell Biol 12:441-53; see also above discussion of vectors containing fusion proteins).

MMLR-CCR or MPHG-CCR may also be expressed as a recombinant protein with one or more additional polypeptide domains added to facilitate protein purification. Such purification facilitating domains include, but are not limited to, metal chelating peptides such as histidine-tryptophan modules that allow purification on immobilized metals (Porath J (1992) Protein Expr Purif 3: 263-281), protein A domains that allow purification on immobilized immunoglobulin, and the domain utilized in the FLAGS extension/affinity purification system (Immunex Corp, Seattle Wash.). The inclusion of a cleavable linker sequence such as Factor XA or enterokinase (Invitrogen, San Diego Calif.) between the purification domain and MMLR-CCR or MPHG-CCR is useful to facilitate purification.

Uses of The Receptors and Genetically Engineered Host Cells Containing the Receptors

The amino acid sequence of MMLR-CCR (SEQ ID NO: 2) is shown in FIG. 1. Based upon its homology to MCP-1 CCR chemokine receptor, MCP-1RB (Charo supra) particularly in the third transmembrane spannng domain which is known to be associated with G protein binding and its presence in a macrophage, 48 hour mixed lymphocyte reaction cDNA library and absence in a normal macrophage MMLR-CCR; and MMLR-CCR appears to be a chemokine receptor associated with leukocyte functioning. The amino acid sequence of MPHG-CCR (SEQ ID NO: 4) is shown in FIG. 6. Based upon it homology to C-C chemokine receptor 3 and MCP-1RA, and its presence in a macrophage library, MPHG-CCR appears to be a chemokine receptor associated with leukocyte functioning.

Accordingly, the present invention provides MMLR-CCR and MPHG-CCR amino acid sequences and genetically engineered host cells that express the receptors to evaluate, screen and identify the naturally occurring ligands, i.e., chemokines, in appropriate cellular supernatants. The chemokine receptors of the present invention and genetically engineered host cells that express the receptors may also be used to identify substances, compounds or synthetic drugs that modulate receptor/ligand binding thereby modulating receptor activation and signalling transduction events. For example, such genetically engineered host cells could be used to screen peptide libraries or organic molecules capable of modulating MMLR-CCR activity.

In an embodiment of the present invention, MMLR-CCR, MPHG-CCR or a variant thereof and/or a cell line that expresses the MMLR-CCR, MPHG-CCR or variant thereof may be used to screen for antibodies, peptides, or other molecules, such as organic or inorganic molecules made by combinatorial chemistry, that act as modulators of monocyte/macrophage infiltration and chemotaxis, thereby identifying a therapeutic capable of modulating the immune response. Anti-receptor antibodies capable of neutralizing the activity of the receptor may be used to inhibit receptor activation, for example, in inflammatory diseases. Synthetic compounds, natural products, and other sources of potentially biologically active materials can be screened in a number of ways deemed to be routine to those of skill in the art. For example, nucleotide sequences encoding an extracellular domain of MMLR-CCR or MPHG-CCR may be expressed in a cell line which can be used for screening of modulators, either agonists or antagonists, of MMLR-CCRA or MPHG-CCR activity, respectively. In one embodiment herein, an MMLR-CCR variant is one containing an isoleucine at residue position 121 instead of valine.

The ability of a test molecule to interfere with chemokine receptor activity or ligand binding may be determined by a measurement of monocyte chemotaxis (Falk et al 1980 J Immunol Methods 33: 239). The activity of a chemokine receptor may also be monitored by measuring other responses such as Ca⁺⁺ flux (Grynkievicz et al (1985) J Biol Chem 260: 3440 and McColl et al (1993) J Immunol 150: 4550-4555) and degranulation and respiratory burst responses (Zachariae et al 1990 J. Exp. Med. 171: 2177-82) and regulation of adhesion molecule expression and cytokine production (Jiang et al 1992 J Immunol 148: 2432-8).

Antibodies

Procedures well known in the art may be used for the production of antibodies to MMLR-CCR or MPHG-CCR polypeptides. Such antibodies include, but are not limited to, polyclonal, monoclonal, chimeric, single chain, Fab fragments and fragments produced by a Fab expression library. Neutralizing antibodies, ie, those which inhibit biological activity of MMLR-CCR or MPHG-CCR polypeptides, are especially preferred for diagnostics and therapeutics.

For the production of antibodies, various hosts including goats, rabbits, rats, mice, etc may be immunized by injection with MMLR-CCR or MPHG-CCR polypeptide or any portion, fragment or oligopeptide which retains immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants which may be employed if purified MMLR-CCR polypeptide is administered to immunologically compromised individuals for the purpose of stimulating systemic defense.

Monoclonal antibodies to MMLR-CCR or MPHG-CCR polypeptide may be prepared using any technique which provides for the production of antibody molecules by continuous cell lines in culture. These include, but are not limited to, the hybridoma technique originally described by Koehler and Milstein (1975, Nature 256: 495-497), the human B-cell hybridoma technique (Kosbor et al (1983) Immunol Today 4: 72; Cote et al (1983) Proc Natl Acad Sci 80: 2026-2030) and the EBV-hybridoma technique (Cole et al (1985) Monoclonal Antibodies and Cancer Therapy, Alan R Liss Inc, pp 77-96). In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81: 6851-6855; Neuberger et al (1984) Nature 312: 604-608; Takeda et al (1985) Nature 314: 452-454). Alternatively, techniques described for the production of single chain antibodies (U.S. Pat. No. 4,946,778) can be adapted to produce specific single chain antibodies.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci 86: 3833-3837), and Winter G and Milstein C (1991; Nature 349: 293-299).

Antibody fragments which contain specific binding sites for MMLR-CCR or MPHG-CCR may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments.

Alternatively, Fab expression libraries may be constructed to allow rapid and easy identification of monoclonal Fab fragments with the desired specificity (Huse W D et al (1989) Science 256: 1275-1281).

MMLR-CCR- or MPHG-CCR-specific antibodies are useful for the diagnosis of conditions and diseases associated with expression of MMLR-CCR or MPHG-CCR polypeptide. A variety of protocols for competitive binding or immunoradiometric assays using either polyclonal or monoclonal antibodies with established specificities are well known in the art. Such immunoassays typically involve the formation of a complex between a chemokine receptor and its specific antibody (or similar receptor-binding molecule) and the measurement of complex formation. A two-site, monoclonal-based immunoassay utilizing monoclonal antibodies specific for two noninterfering epitopes on a specific MMLR-CCR or MPHG-CCR protein is preferred, but a competitive binding assay may also be employed. These assays are described in Maddox D E et al (1983, J Exp Med 158: 1211).

Diagnostic Assays Using Receptor Specific Antibodies

Anti-MMLR-CCR or MPHG-CCR antibodies are useful for the diagnosis of conditions related to receptor activation, such as, infection, inflammation, tumorigenesis, abnormal proliferation of cells and cardiovascular disease. Diagnostic assays for the chemokine receptors include methods utilizing the antibody and a label to detect a MMLR-CCR or MPHG-CCR polypeptide in human body fluids, cells, tissues or sections or extracts of such tissues. The polypeptides and antibodies of the present invention may be used with or without modification. Frequently, the polypeptides and antibodies will be labeled by joining them, either covalently or noncovalently, with a reporter molecule. A wide variety of reporter molecules are known to those of skill in the art.

A variety of protocols for measuring a MMLR-CCR or MPHG-CCR polypeptide, using either polyclonal or monoclonal antibodies specific for the respective protein are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes on a MMLR-CCR or MPHG-CCR polypeptide is preferred, but a competitive binding assay may be employed. These assays are described, among other places, in Maddox, D E et al (1983, J Exp Med 158: 1211).

In order to provide a basis for the diagnosis of disease, normal or standard values for MMLR-CCR or MPHG-CCR polypeptide expression are established. This is accomplished by combining body fluids or cell extracts taken from normal subjects, either animal or human, with antibody to a MMLR-CCR or MPHG-CCR polypeptide under conditions suitable for complex formation which are well known in the art. The amount of standard complex formation can be quantified by comparing it with a dilution series of positive controls where a known amount of antibody is combined with known concentrations of a purified MMLR-CCR or MPHG-CCR polypeptide. Then, standard values obtained from normal samples may be compared with values obtained from samples from subjects potentially affected by a disorder or disease related to a chemokine receptor polypeptide expression. Deviation between standard and subject values establishes the presence of the disease state.

Drug Screening

An MMLR-CCR polypeptide or MPHG-CCR polypeptide, its immunogenic fragments or oligopeptides thereof can be used for screening therapeutic compounds in any of a variety of drug screening techniques. The fragment employed in such a test may be free in solution, affixed to a solid support, borne on a cell surface, or located intracellularly. The abolition of activity or the formation of binding complexes, between a receptor polypeptide and the agent being tested, may be measured. Accordingly, the present invention provides a method for screening a plurality of compounds for specific binding affinity with a MMLR-CCR, MPHG-CCR or a fragment thereof, comprising providing a plurality of compounds; combining a chemokine receptor of the present invention or a fragment thereof with each of a plurality of compounds for a time sufficient to allow binding under suitable conditions; and detecting binding of the chemokine receptor, or fragment thereof, to each of the plurality of compounds, thereby identifying the compounds which specifically bind the chemokine receptor. In such an assay, the plurality of compounds may be produced by combinatorial chemistry techniques known to those of skill in the art. In one embodiment of the present invention the MMLR-CCR or MPHG-CCR oligopeptide is obtained from an extracellular binding domain.

Another technique for drug screening provides for high throughput screening of compounds having suitable binding affinity to a MMLR-CCR or MPHG-CCR polypeptide and is described in detail in Geysen, European Patent Application 84/03564, published on Sep. 13, 1984, incorporated herein by reference. In summary, large numbers of different small peptide test compounds are synthesized on a solid substrate, such as plastic pins or some other surface. The peptide test compounds are reacted with MMLR-CCR or MPHG-CCR fragments and washed. A bound chemokine receptor of the present invention is then detected by methods well known in the art. A purified MMLR-CCR or MPHG-CCR can also be coated directly onto plates for use in the aforementioned drug screening techniques. Alternatively, non-neutralizing antibodies can be used to capture the peptide and immobilize it on a solid support.

This invention also contemplates the use of competitive drug screening assays in which neutralizing antibodies capable of binding MMLR-CCR or MPHG-CCR specifically compete with a test compound for binding MMLR-CCR or MPHG-CCR. In this manner, the antibodies can be used to detect the presence of any peptide which shares one or more antigenic determinants with MMLR-CCR or MPHG-CCR, respectively.

Uses of Receptor Polynucleotide

An MMLR-CCR or MPHG-CCR polynucleotide, or any part thereof, provides the basis for diagnostic and/or therapeutic compounds. For diagnostic purposes, MMLR-CCR or MPHG-CCR polynucleotide sequences are used to detect and quantitate gene expression in conditions, disorders and diseases in which their activity may be implicated, for example, in infection, inflammation, tumorigenesis, proliferative disease and cardiovascular disease. For therapeutic purposes, MMLR-CCR or MPHG-CCR antisense molecules are administered to individuals in conditions where it is desirable to down-regulate the presence of the receptor on the cell surface thereby inhibiting the activity of MMLR-CCR or MPHG-CCR, respectively. Alternatively, for therapeutic purposes, sense polynucleotide sequences of MMLR-CCR or MPHG-CCR are administered to individuals in conditions where it is desirable to enhance signal transduction events.

Included in the scope of the invention are oligonucleotide sequences, antisense RNA and DNA molecules and ribozymes, which function to destabilize MMLR-CCR or MPHG-CCR mRNA or inhibit translation of a MMLR-CCR or MPHF-CCR.

Another aspect of the subject invention is to provide for nucleic acid hybridization or PCR probes which can detect polynucleotide sequences, including genomic sequences, encoding mmlr-ccr or closely related molecules, such as alleles. The specificity of the probe, i.e, whether it is derived from a highly conserved, conserved or non-conserved region or domain, and the stringency of the hybridization or amplification (high, intermediate or low) determines whether the probe identifies only naturally occurring mmlr-ccr or related sequences. Probes for the detection of related nucleic acid sequences are selected from conserved or highly conserved nucleotide regions of known chemokine receptors, for example, the G protein binding domains. For the detection of identical nucleic acid sequences, or where maximum specificity is desired, such as in a diagnostic test, nucleic acid probes are selected from the non-conserved nucleotide regions or unique regions of mmlr-ccr polynucleotides. As used herein, the term “non-conserved nucleotide region” refers to a nucleotide region that is unique to the mmlr-ccr and mphg-ccr disclosed herein and does not occur in related chemokine receptors.

Diagnostic Uses of Receptor Polynucleotide

An MMLR-CCR or MPHG-CCR encoding polynucleotide sequence is used for the diagnosis of diseases associated with receptor activation, such as infection, inflammation, tumorigenesis, and cardiovascular disease. For example, polynucleotide sequences encoding MMLR-CCR or MPHG-CCR are used in hybridization or PCR assays of tissues from biopsies or autopsies or biological fluids, such as serum, synovial fluid or tumor biopsy, to detect abnormalities in MMLR-CCR or MPHG-CCR expression. Such qualitative or quantitative methods include Southern or northern analysis, dot blot or other membrane-based technologies; PCR technologies; dip stick, pin or chip technologies; and ELISA or other multiple sample format technologies. All of these techniques are well known in the art and are in fact the basis of many commercially available diagnostic kits.

Such assays are tailored to evaluate the efficacy of a particular therapeutic treatment regime and are used in animal studies, in clinical trials, or in monitoring the treatment of an individual patient. For disease diagnosis, a normal or standard profile for MMLR-CCR or MPHG-CCR expression is first established. This is accomplished by combining body fluids or cell extracts taken from normal subjects, either animal or human, with MMLR-CCR or MPHG-CCR or a portion thereof, under conditions suitable for hybridization or amplification. Standard hybridization is quantified by comparing the values obtained for normal subjects with a dilution series of positive controls run in the same experiment where a known amount of purified MMLR-CCR or MPHG-CCR is used. Standard values obtained from normal samples are compared with values obtained from samples from subjects potentially affected by a disorder or disease related to MMLR-CCR or MPHG-CCR expression. Deviation between standard and subject values establishes the presence of the disease state. If disease is established, an existing therapeutic agent is administered, and treatment profile or values may be generated. Finally, the assay may be repeated on a regular basis to evaluate whether the values progress toward or return to the normal or standard pattern. Successive treatment profiles may be used to show the efficacy of treatment over a period of several days or several months.

PCR as described in U.S. Pat. Nos. 4,683,195; 4,800,195; and 4,965,188 provides additional uses for oligonucleotides based upon the MMLR-CCR or MPHG-CCR sequence. Such oligomers are generally chemically synthesized, but they are generated enzymatically or produced from a recombinant source. Oligomers generally comprise two nucleotide sequences, one with sense orientation (5′->3′) and one with antisense (3′<-5′) employed under optimized conditions for identification of a specific gene or condition. The same two oligomers, nested sets of oligomers, or even a degenerate pool of oligomers are employed under less stringent conditions for detection and/or quantitation of closely related DNA or RNA sequences.

Additionally methods to quantitate the expression of a particular molecule include radiolabeling (Melby P C et al 1993 J Immunol Methods 159: 235-44) or biotinylating (Duplaa C et al 1993 Anal Biochem 229-36) nucleotides, coamplification of a control nucleic acid, and standard curves onto which the experimental results are interpolated. Quantitation of multiple samples is speeded up by running the assay in an ELISA format where the an oligomer of interest is presented in various dilutions and a spectrophotometric or colorimetric response gives rapid quantitation.

Therapeutic Uses of an MMLR-CCR or MPHG-CCR Polynucleotide

An MMLR-CCR or MPHG-CCR antisense molecule can provide the basis for treatment of various abnormal conditions related to receptor activation such as infection, inflammation, abnormal proliferation of cell, tumogenisis and cardiovascular disease, such as atherosclerosis, where it would be desirable to downregulate the presence of the receptor, thereby inhibiting MMLR-CCR or MPHG-CCR activity. Alternatively, polynucleotide sequences encoding MMLR-CCR or MPHG-CCR may provide the basis for gene therapy in the treatment of various abnormal conditions where it is desirable to up regulate the receptors thereby enhancing the immune response.

Expression vectors derived from retroviruses, adenovirus, herpes or vaccinia viruses, or from various bacterial plasmids, are used for delivery of recombinant MMLR-CCR or MPHG-CCR sense or antisense molecules to the targeted cell population. Methods which are well known to those skilled in the art can be used to construct recombinant vectors containing MMLR-CCR or MPHG-CCR related nucleotide sequences. See, for example, the techniques described in Maniatis et al (supra) and Ausubel et al(supra). Alternatively, recombinant MMLR-CCR or MPHG-CCR can be delivered to target cells in liposomes.

The full length cDNA sequence and/or its regulatory elements enable researchers to use a MMLR-CCR or MPHG-CCR as a tool in sense (Youssoufian H and H F Lodish 1993 Mol Cell Biol 13: 98-104) or antisense (Eguchi et al (1991) Annu Rev Biochem 60 : 631-652) investigations of gene function. Oligonucleotides, designed from the cDNA or control sequences obtained from the genomic DNA can be used in vitro or in vivo to inhibit expression. Such technology is now well known in the art, and sense or antisense oligonucleotides or larger fragments can be designed from various locations along the coding or control regions.

Additionally, MMLR-CCR or MPHG-CCR expression can be modulated by transfecting a cell or tissue with expression vectors which express high levels of a MMLR-CCR or MPHG-CCR fragment in conditions where it would be preferable to inhibit MMLR-CCR or MPHG-CCR activity. Such constructs can flood cells with untranslatable sense or antisense sequences which compete for binding with the naturally occurring receptor. Even in the absence of integration into the DNA, such vectors may continue to transcribe RNA molecules until all copies of the vector are disabled by endogenous nucleases. Such transient expression may last for a month or more with a non-replicating vector (Mettler I, personal communication) and even longer if appropriate replication elements are part of the vector system.

Modifications of gene expression can be obtained by designing antisense sequences to the control regions of the MMLR-CCR or MPHG-CCR gene, such as the promoters, enhancers, and introns. Oligonucleotides derived from the transcription initiation site, eg, between −10 and +10 regions of the leader sequence, are preferred. Antisense RNA and DNA molecules may also be designed to block translation of mRNA by preventing the transcript from binding to ribosomes. Similarly, inhibition can be achieved using Hogeboom base-pairing methodology, also known as “triple helix” base pairing. Triple helix pairing compromises the ability of the double helix to open sufficiently for the binding of polymerases, transcription factors, or regulatory molecules.

Ribozymes are enzymatic RNA molecules capable of catalyzing the specific cleavage of RNA. The mechanism of ribozyme action involves sequence specific hybridization of the ribozyme molecule to complementary target RNA, followed by a endonucleolytic cleavage. Within the scope of the invention are engineered hammerhead motif ribozyme molecules that specifically and efficiently catalyze endonucleolytic cleavage of MMLR-CCR or MPHG-CCR RNA sequences.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for secondary structural features which may render the oligonucleotide sequence inoperable. The suitability of candidate targets may also be evaluated by testing accessibility to hybridization with complementary oligonucleotides using ribonuclease protection assays.

Both antisense RNA and DNA molecules and ribozymes of the invention may be prepared by any method known in the art for the synthesis of RNA molecules. These include techniques for chemically synthesizing oligonucleotides such as solid phase phosphoramidite chemical synthesis. Alternatively, RNA molecules may be generated by in vitro or in vivo transcription of DNA sequences encoding the antisense RNA molecule. Such DNA sequences may be incorporated into a wide variety of vectors with suitable RNA polymerase promoters such as T7 or SP6. Alternatively, antisense cDNA constructs that synthesize antisense RNA constitutively or inducibly can be introduced into cell lines, cells or tissues.

DNA molecules may be modified to increase intracellular stability and half-life. Possible modifications include, but are not limited to, the addition of flanking sequences of the 5′ and/or 3′ ends of the molecule or the use of phosphorothioate or 2′ O-methyl rather than phosphodiesterase linkages within the backbone of the molecule.

Methods for introducing vectors into cells or tissue include those methods discussed infra. In addition, several of these transformation or transfection methods are equally suitable for ex vivo therapy,

Furthermore, the MMLR-CCR or MPHG-CCR polynucleotide sequences disclosed herein may be used in molecular biology techniques that have not yet been developed, provided the new techniques rely on properties of nucleotide sequences that are currently known, including but not limited to such properties as the triplet genetic code and specific base pair interactions.

Detection and Mapping of Polynucleotide Sequences Related to MMLR-CCR OR MPHG-CCR

The nucleic acid sequence for MMLR-CCR or MPHG-CCR can also be used to generate hybridization probes as previously described, for mapping the endogenous genomic sequence. The sequence may be mapped to a particular chromosome or to a specific region of the chromosome using well known techniques. These include in situ hybridization to chromosomal spreads (Verma et al (1988) Human Chromosomes: A Manual of Basic Techniques, Pergamon Press, New York City), flow-sorted chromosomal preparations, or artificial chromosome constructions such as YACs, bacterial artificial chromosomes (BACs), bacterial P1 constructions or single chromosome cDNA libraries.

In situ hybridization of chromosomal preparations and physical mapping techniques such as linkage analysis using established chromosomal markers are invaluable in extending genetic maps. Examples of genetic maps can be found in Science (1995; 270: 410f and 1994; 265: 1981f). Often the placement of a gene on the chromosome of another mammalian species may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once a disease or syndrome, such as ataxia telangiectasia (AT), has been crudely localized by genetic linkage to a particular genomic region, for example, AT to 11q22-23 (Gatti et al (1988) Nature 336: 577-580), any additional sequences mapping to that area may represent associated or regulatory genes for further investigation. The nucleotide sequence of the subject invention may also be used to detect differences in the chromosomal location due to translocation, inversion, etc between normal, carrier or affected individuals.

Pharmaceutical Compositions

The present invention relates to pharmaceutical compositions which may comprise nucleotides, proteins, antibodies, antagonists, or inhibitors, or agonists of MMLR-CCR or MPHG-CCR alone or in combination with at least one other agent, such as stabilizing compound, which may be administered in any sterile, biocompatible pharmaceutical carrier, including, but not limited to, saline, buffered saline, dextrose, and water. Any of these molecules can be administered to a patient alone, or in combination with other agents, drugs or hormones, in pharmaceutical compositions where it is mixed with excipient(s) or pharmaceutically acceptable carriers. In one embodiment of the present invention, the pharmaceutically acceptable carrier is pharmaceutically inert.

Administration of Pharmaceutical Compositions

Administration of pharmaceutical compositions is accomplished orally or parenterally. Methods of parenteral delivery include topical, intra-arterial (directly to the tumor), intramuscular, subcutaneous, intramedullary, intrathecal, intraventricular, intravenous, intraperitoneal, or intranasal administration. In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of “Remington's Pharmaceutical Sciences” (Maack Publishing Co, Easton Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, ie, dosage.

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hanks's solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

Manufacture and Storage

The pharmaceutical compositions of the present invention may be manufactured in a manner that known in the art, eg, by means of conventional mixing, dissolving, granulating, dragee-making, levigating, emulsifying, encapsulating, entrapping or lyophilizing processes.

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

After pharmaceutical compositions comprising a compound of the invention formulated in a acceptable carrier have been prepared, they can be placed in an appropriate container and labeled for treatment of an indicated condition. For administration of HCAP, such labeling would include amount, frequency and method of administration.

Therapeutically Effective Dose

Pharmaceutical compositions suitable for use in the present invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, eg, of neoplastic cells, or in animal models, usually mice, rabbits, dogs, or pigs. The animal model is also used to achieve a desirable concentration range and route of administration. Such information can then be used to determine useful doses and routes for administration in humans.

A therapeutically effective dose refers to that amount of protein or its antibodies, antagonists, or inhibitors or agonists which ameliorate the symptoms or condition. Therapeutic efficacy and toxicity of such compounds can be determined by standard pharmaceutical procedures in cell cultures or experimental animals, eg, ED50 (the dose therapeutically effective in 50% of the population) and LD50 (the dose lethal to 50% of the population). The dose ratio between therapeutic and toxic effects is the therapeutic index, and it can be expressed as the ratio, ED50/LD50. Pharmaceutical compositions which exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors which may be taken into account include the severity of the disease state, eg, tumor size and location; age, weight and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature. See U.S. Pat. Nos. 4,657,760 ; 5,206,344; or 5,225,212. Those skilled in the art will employ different formulations for nucleotides than for proteins or their inhibitors. Similarly, delivery of polynucleotides or polypeptides will be specific to particular cells, conditions or locations, for example.

It is contemplated that antagonists or agonists of MMLR-CCR or MPHG-CCR HCAP can be delivered in a suitable formulation to individuals having conditions where it is desirable to inhibit or enhance, respectively, chemokine receptor activity.

These examples are provided by way of illustration and are not included for the purpose of limiting the invention.

### EXAMPLES

I Construction of Mmlr-Ccr Library and Isolation of Cdna Clones

By way of example, construction of the MMLR2DT01 cDNA library is described. The MPHGNOT03 library was constructed using similar methodology.

The normal peripheral blood macrophages used for this library were obtained from two 24-year-old, Caucasian males. This library represents a mixture of allogeneically stimulated human macrophage populations obtained from Ficoll/Hypaque purified buffy coats. The cells from the two different donors (not typed for HLA alleles) were incubated at a density of 1×10⁶/ml, cultured for 48 hours in DME containing 10% human serum.

After incubation, macrophages mostly adhered to the plastic surface of the petri dish, whereas most other cell types, B and T lymphocytes, remained in solution. The DME was decanted from the wells and the wells were washed with phosphate buffered saline (PBS). Macrophages were released from the plastic surface by gently scraping the petri dishes in PBS/1 mM EDTA. Macrophages were lysed immediately in buffer containing guanidinium isothiocyanate.

The lysate was extracted twice with a mixture of phenol and chloroform, pH 8.0 and centrifuged over a CsCl cushion using an Beckman SW28 rotor in a L8-70M Ultracentrifuge (Beckman Instruments). The RNA was precipitated using 0.3 M sodium acetate and 2.5 volumes of ethanol, resuspended in water and DNase treated for 15 min at 37° C. The total RNA was isolated using the Qiagen Oligotex kit (QIAGEN Inc, Chatsworth Calif.). It must be noted that some contaminating T and B lymphocytes may also have been present.

The poly A⁺ RNA was used in the SuperScript Plasmid System for cDNA Synthesis and Plasmid Cloning (catalogue #18248-013; Gibco BRL, Gaithersburg Md.) with the recommended protocol. cDNAs were fractionated on a Sepharose CL4B column (catalog #275105, Pharmacia, and those cDNAs exceeding 400 bp were ligated into pSport I. The plasmid was transformed into chemically competent DH5 host cells (Gibco BRL).

Plasmid DNA was released from the cells and purified using the Miniprep Kit (Catalogue # 77468; Advanced Genetic Technologies Corporation, Gaithersburg Md.). This kit consists of a 96 well block with reagents for 960 purifications. The recommended protocol was employed except for the following changes: 1) the 96 wells were each filled with only 1 ml of sterile Terrific Broth (Catalog # 22711, LIFE TECHNOLOGIES™, Gaithersburg Md.) with carbenicillin at 25 mg/L and glycerol at 0.4%; 2) the bacteria were cultured for 24 hours after the wells were inoculated and then lysed with 60 μl of lysis buffer; 3) a centrifugation step employing the Beckman GS-6R @2900 rpm for 5 min was performed before the contents of the block were added to the primary filter plate; and 4) the optional step of adding isopropanol to TRIS buffer was not routinely performed. After the last step in the protocol, samples were transferred to a Beckman 96-well block for storage.

Alternative methods of purifying plasmid DNA include the use of MAGIC MINIPREPS™ DNA Purification System (Catalogue #A7100, Promega, Madison Wis.)or QIAwell™ -8 Plasmid, QIAwell PLUS DNA and QIAwell ULTRA DNA Purification Systems (QIAGEN® Chatsworth Calif.).

The cDNAs were sequenced by the method of Sanger F and A R Coulson (1975; J Mol Biol 94: 441f), using a Hamilton Micro Lab 2200 (Hamilton, Reno Nev.) in combination with four Peltier Thermal Cyclers (PTC200 from M J Research, Watertown Mass.) and Applied Biosystems 377 or 373 DNA Sequencing Systems (Perkin Elmer) and reading frame was determined.

II Homology Searching of cDNA Clones and Their Deduced Proteins

Each cDNA was compared to sequences in GenBank using a BLAST search (Basic Local Alignment Search Tool; Altschul S F (1993) J. Mol. Evol. 36: 290-300; Altschul S F et al (1990) J. Mol. Biol. 215: 403-410). This method identified Incyte Clone 478861 as a non-exact match to human MCP-1RB receptor (NCBI GI 472557) Charo supra and Incyte Clone 442279 as being a non-exact match to C-C chemokine receptor 3 and MCP-1RA (Charo supra).

BLAST was used to search for local sequence alignments . BLAST produces alignments of both nucleotide and amino acid sequences to determine sequence similarity. Because of the local nature of the alignments, BLAST is especially useful in determining exact matches and in identifying homologs. BLAST is useful for matches which do not contain gaps. The fundamental unit of BLAST algorithm output is the High-scoring Segment Pair (HSP).

An HSP consists of two sequence fragments of arbitrary but equal lengths whose alignment is locally maximal and for which the alignment score meets or exceeds a threshold or cutoff score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

III DETERMINATION OF READING FRAME OF cDNA CLONE

The reading frame of individual cDNA clones obtained from the MMLR2DT01 library was obtained by analyzing the polynucleotide sequences for the presence of start (ATG, GTG, etc.) and stop codons (TGA, TAA, TAG). Typically, one frame will continue throughout the major portion of all of a cDNA sequence and the other two pending frames tend to contain numerous stop codons. Algorithms for determining reading frame have been developed which analyze the occurrence of individual nucleotide bases of each putative codon triplet (e.g., Fickett, J. W. Nucleic Acids Research, 10, 5303 (1982)). Coding DNA tends to contain predominantly certain nucleotides within certain triplet periodicities, such as a significant preference for pyrimidines in the third codon position. These algorithms have been incorporated into widely available software and are used to determine coding potential (and frame) of a given stretch of DNA. This algorithm-derived information, combined with start/stop codon information, was used to determine proper frame of individual clones within the library with a high degree of certainty, thus permitting the correct reading frame alignment with appropriate expression vehicles.

IV Extension of MMLR-CCR OR MPHG-CCR to Recover Regulatory Elements

The nucleic acid sequence of MMLR-CCR or MPHG-CCR is used to design oligonucleotide primers for obtaining full length sequences from genomic libraries. One primer is synthesized to initiate extension in the antisense direction (XLR) and the other is synthesized to extend sequence in the sense direction (XLF). The primers allow the known MMLR-CCR or MPHG-CCR sequence to be extended “outward” generating amplicons containing new, unknown nucleotide sequence for the control region of interest. The initial primers are designed from the cDNA using Oligo 4.0 (National Biosciences Inc, Plymouth Minn.), or another appropriate program, to be 22-30 nucleotides in length, to have a GC content of 50% or more, and to anneal to the target sequence at temperatures about 68°-72° C. Any stretch of nucleotides which would result in hairpin structures and primer-primer dimerizations is avoided.

A human genomic library is used to extend and amplify 5′ upstream sequence. If necessary, a second set of primers is designed to further extend the known region. By following the instructions for the XL-PCR kit (Perkin Elmer) and thoroughly mixing the enzyme and reaction mix, high fidelity amplification is obtained. Beginning with 40 pmol of each primer and the recommended concentrations of all other components of the kit, PCR is performed using the Peltier Thermal Cycler (PTC200; M J Research, Watertown Mass.) and the following parameters:

| | |
|---|---|
| Step 1 | 94° C. for 1 mm (initial denaturation) |
| Step 2 | 65° C. for 1 min |
| Step 3 | 68° C. for 6 min |
| Step 4 | 94° C. for 15 sec |
| Step 5 | 65° C. for 1 min |
| Step 6 | 68° C. for 7 min |
| Step 7 | Repeat step 4-6 for 15 additional cycles |
| Step 8 | 94° C. for 15 sec |
| Step 9 | 65° C. for 1min |
| Step 10 | 68° C. for 7:15 min |
| Step 11 | Repeat step 8-10 for 12 cycles |
| Step 12 | 72° C. for 8 min |
| Step 13 | 4° C. (and holding) |

A 5-10 μl aliquot of the reaction mixture is analyzed by electrophoresis on a low concentration (about 0.6-0.8%) agarose mini-gel to determine which reactions were successful in extending the sequence. The largest products or bands are selected and cut out of the gel. Further purification involves using a commercial gel extraction method such as QIAQuick™ (QIAGEN Inc). After recovery of the DNA, Klenow enzyme is used to trim single-stranded, nucleotide overhangs creating blunt ends which facilitate religation and cloning.

After ethanol precipitation, the products are redissolved in 13 μl of ligation buffer, 1 μl T4-DNA ligase (15 units) and 1 μl T4 polynucleotide kinase are added, and the mixture is incubated at room temperature for 2-3 hours or overnight at 16° C. Competent E. coli cells (in 40 μl of appropriate media) are transformed with 3 μl of ligation mixture and cultured in 80 μl of SOC medium (Sambrook J et al, supra). After incubation for one hour at 37° C., the whole transformation mixture is plated on Luria Bertani (LB)-agar (Sambrook J et al, supra) containing 2× Carb. The following day, several colonies are randomly picked from each plate and cultured in 150 μl of liquid LB/2×Carb medium placed in an individual well of an appropriate, commercially-available, sterile 96-well microtiter plate. The following day, 5 μl of each overnight culture is transferred into a non-sterile 96-well plate and after dilution 1:10 with water, 5 μl of each sample is transferred into a PCR array.

For PCR amplification, 18 μl of concentrated PCR reaction mix (3.3×) containing 4 units of rTth DNA polymerase, a vector primer and one or both of the gene specific primers used for the extension reaction are added to each well. Amplification is performed using the following conditions:

| | |
|---|---|
| Step 1 | 94° C. for 60 sec |
| Step 2 | 94° C. for 20 sec |
| Step 3 | 55° C. for 30 sec |
| Step 4 | 72° C. for 90 sec |
| Step 5 | Repeat steps 2-4 for an additional 29 cycles |
| Step 6 | 72° C. for 180 sec |
| Step 7 | 4° C. (and holding) |

Aliquots of the PCR reactions are run on agarose gels together with molecular weight markers. The sizes of the PCR products are compared to the original partial cDNAs, and appropriate clones are selected, ligated into plasmid and sequenced.

V Labeling of Hybridization Probes

Hybridization probes derived from SEQ ID NO: 1 or SEQ ID NO: 3 are employed to screen cDNAs, mRNAs or genomic DNAs. Although the labeling of oligonucleotides, consisting of about 20 base-pairs, is specifically described, essentially the same procedure is used with larger cDNA fragments. Oligonucleotides are labeled by combining 50 pmol of each oligomer and 250 mCi of [γ-³²P] adenosine triphosphate (Amersham, Chicago Ill.) and T4 polynucleotide kinase (DuPont NEN®, Boston Mass.). The labeled oligonucleotides are purified with Sephadex G-25 super fine resin column (Pharmacia). A portion containing 10⁷ counts per minute of each is used in a typical membrane based hybridization analysis of human genomic DNA digested with one of the following endonucleases (Ase I, Bgl II, EcoR I, Pst I, Xba 1, or Pvu II; DuPont NEN®).

The DNA from each digest is fractionated on a 0.7 percent agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under increasingly stringent conditions up to 0.1×saline sodium citrate and 0.5% sodium dodecyl sulfate. After XOMAT AR™ film (Kodak, Rochester N.Y.) is exposed to the blots in a Phosphoimager cassette (Molecular Dynamics, Sunnyvale Calif.) for several hours, hybridization patterns are compared visually.

VI Antisense Molecules

The MMLR-CCR or MPHG-CCR sequence, or any part thereof, is used to inhibit in vivo or in vitro expression of endogenous MMLR-CCR or MPHG-CCR, respectively. Although use of antisense oligonucleotides, consisting of about 20 base-pairs, is specifically described, essentially the same procedure is used with larger cDNA fragments. An oligonucleotide based on the coding sequence of MMLR-CCR or MPHG-CCR is used to inhibit expression of endogenous MMLR-CCR or MPHG-CCR. Using Oligo 4.0, the complementary oligonucleotide is designed from the conserved 5′ sequence and used to inhibit either transcription, by preventing promoter binding to the upstream nontranslated sequence, or translation of an MMLR-CCR or MPHG-CCR transcript by preventing the ribosome from binding to the mRNA.

VII Production of MMLR-CCR OR MPHG-CCR Specific Antibodies

For production of polyclonal antibodies, the deduced amino acid sequence of MMLR-CCR or MPHG-CCR is analyzed using DNASTAR software (DNASTAR Inc) to determine regions of high immunogenicity and a corresponding oligopeptide is synthesized and used to raise antibodies in rabbits. Analysis to select appropriate epitopes, such as those near the C-terminus or in adjacent hydrophilic regions is described by Ausubel F M et al (supra). An oligopeptide of about 15 residues in length is synthesized using an ABI Peptide Synthesizer Model 431A (Perkin Elmer, Norwalk, Conn.) using fmoc-chemistry, and coupled to keyhole limpet hemocyanin (KLH, Sigma) by reaction with M-maleimidobenzoyl-N-hydroxysuccinimide ester (MBS; Ausubel F M et al, supra). Rabbits are immunized with the oligopeptide-KLH complex in complete Freund's adjuvant. The resulting antisera are tested for antipeptide activity, for example, by binding the peptide to plastic, blocking with 1% BSA, reacting with rabbit antisera, washing, and reacting with radioiodinated, goat anti-rabbit IgG.

VIII Purification of MMLR-CCR Or MPHG-CCR Using Specific Antibodies

Endogenous or recombinant MMLR-CCR or MPHG-CCR can be purified by immunoaffinity chromatography using antibodies specific for MMLR-CCR or MPHG-CCR. An immunoaffinity column is constructed by covalently coupling MMLR-CCR or MPHG-CCR antibody to an activated chromatographic resin such as CnBr-activated Sepharose (Pharmacia Biotech). After the coupling, the resin is blocked and washed according to the manufacturer's instructions.

Media containing MMLR-CCR or MPHG-CCR is passed over the immunoaffinity column, and the column is washed under conditions that allow the preferential absorbance of MMLR-CCR or MPHG-CCR (eg, high ionic strength buffers in the presence of detergent). The column is eluted under conditions that disrupt antibody/MMLR-CCR or MPHG-CCR binding (eg, a buffer of pH 2-3 or a high concentration of a chaotrope such as urea or thiocyanate ion), and MMLR-CCR or MPHG-CCR is collected.

IX Identification of Molecules Which Interact with MMLR-CCR Or MPHG-CCR

MMLR-CCR or MPHG-CCR, or a biologically active fragment thereof, is labeled with ¹²⁵¹I Bolton-Hunter reagent (Bolton, A E and Hunter, W M (1973) Biochem J 133: 529). Candidate small molecules previously arrayed in the wells of a 96 well plate are incubated with the labeled MMLR-CCR or MPHG-CCR, washed and any wells with labeled MMLR-CCR or MPHG-CCR complex are assayed. Data obtained using different concentrations of MMLR-CCR or MPHG-CCR are used to calculate values for the number, affinity, and association of MMLR-CCR or MPHG-CCR with the candidate molecules.

X. Northern Analysis

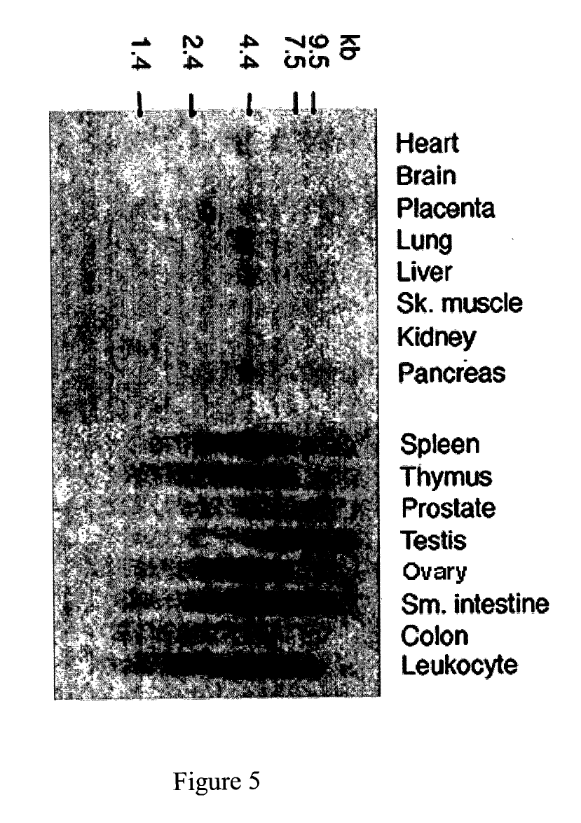

Northern analysis of various tissues obtained from Clontech Labs using the 0.6 kb SalI/NotI fragment from Incyte Clone 478861, which encodes MMLR-CCR, was performed using 0.2× SSC / 0.1% SDS washes. The results showed that a 4.0 kb major transcript exists in all of the 16 tissues represented, see FIG. 5, which include normal heart, brain, placenta, lung, liver skeletal muscle, kidney, pancreas, spleen, thymus, prostate, testis, ovary, small intestine, colon and leukocyte. As illustrated in FIG. 5, the transcript was most abundant in lung, spleen, thymus, ovary, small intestine and peripheral blood leukocyte preparations. A smaller transcript at 2.5 kb is observed in placenta.

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

What is claimed is:

1. An isolated polypeptide selected from the group consisting of:
a) a polypeptide comprising an amino acid sequence selected from the group consisting of SEQ ID NO: 2 and SEQ ID NO: 4,
b) a polypeptide comprising a naturally occurring amino acid sequence at least 90% identical to an amino acid sequence selected from the group consisting of SEQ ID NO: 2 and SEQ ID NO: 4,
c) a biologically active fragment of a polypeptide having an amino acid sequence selected from the group consisting of SEQ ID NO: 2 and SEQ ID NO: 4, and
d) an immunogenic fragment of a polypeptide having an amino acid sequence selected from the group consisting of SEQ ID NO: 2 and SEQ ID NO: 4.

2. An isolated polypeptide of claim 1 comprising an amino acid sequence selected from the group consisting of SEQ ID NO: 2 and SEQ ID NO: 4.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO: 1 and SEQ ID NO: 3.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO: 2 and SEQ ID NO: 4.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO: 1 and SEQ ID NO: 3,
b) a polynucleotide comprising a naturally occurring polynucleotide sequence at least 90% identical to a polynucleotide sequence selected from the group consisting of SEQ ID NO: 1 and SEQ ID NO: 3,
c) a polynucleotide complementary to a polynucleotide of a),
d) a polynucleotide complementary to a polynucleotide of b), and
e) an RNA equivalent of a)-d).

13. An isolated polynucleotide comprising at least 60 contiguous nucleotides of a polynucleotide of claim 12.

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. A method of claim 14, wherein the probe comprises at least 60 contiguous nucleotides.

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO: 2 and SEQ ID NO: 4.

19. A method for treating a disease or condition associated with decreased expression of functional MMLR-CCR or MPHG-CCR, comprising administering to a patient in need of such treatment the composition of claim 17.

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. A composition comprising an agonist compound identified by a method of claim 20 and a pharmaceutically acceptable excipient.

22. A method for treating a disease or condition associated with decreased expression of functional MMLR-CCR or MPHG-CCR, comprising administering to a patient in need of such treatment a composition of claim 21.

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. A composition comprising an antagonist compound identified by a method of claim 23 and a pharmaceutically acceptable excipient.

25. A method for treating a disease or condition associated with overexpression of functional MMLR-CCR or MPHG-CCR, comprising administering to a patient in need of such treatment a composition of claim 24.

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. A method of screening for a compound that modulates the activity of the polypeptide of claim 1, the method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30. A diagnostic test for a condition or disease associated with the expression of MMLR-CCR or MPHG-CCR in a biological sample, the method comprising:
a) combining the biological sample with an antibody of claim 11, under conditions suitable for the antibody to bind the polypeptide and form an antibody:polypeptide complex, and
b) detecting the complex, wherein the presence of the complex correlates with the presence of the polypeptide in the biological sample.

31. The antibody of claim 11, wherein the antibody is:
a) a chimeric antibody,
b) a single chain antibody,
c) a Fab fragment,
d) a F(ab′)₂ fragment, or
e) a humanized antibody.

32. A composition comprising an antibody of claim 11 and an acceptable excipient.

33. A method of diagnosing a condition or disease associated with the expression of MMLR-CCR or MPHG-CCR in a subject, comprising administering to said subject an effective amount of the composition of claim 32.

34. A composition of claim 32, wherein the antibody is labeled.

35. A method of diagnosing a condition or disease associated with the expression of MMLR-CCR or MPHG-CCR in a subject, comprising administering to said subject an effective amount of the composition of claim 34.

36. A method of preparing a polyclonal antibody with the specificity of the antibody of claim 11, the method comprising:
a) immunizing an animal with a polypeptide consisting of an amino acid sequence selected from the group consisting of SEQ ID NO: 2 and SEQ ID NO: 4 or an immunogenic fragment thereof, under conditions to elicit an antibody response,
b) isolating antibodies from said animal, and
c) screening the isolated antibodies with the polypeptide, thereby identifying a polyclonal antibody which specifically binds to a polypeptide comprising an amino acid sequence selected from the group consisting of SEQ ID NO: 2 and SEQ ID NO: 4.

37. A polyclonal antibody produced by a method of claim 36.

38. A composition comprising the polyclonal antibody of claim 37 and a suitable carrier.

39. A method of making a monoclonal antibody with the specificity of the antibody of claim 11, the method comprising:
a) immunizing an animal with a polypeptide consisting of an amino acid sequence selected from the group consisting of SEQ ID NO: 2 and SEQ ID NO: 4, or an immunogenic fragment thereof, under conditions to elicit an antibody response,
b) isolating antibody producing cells from the animal,
c) fusing the antibody producing cells with immortalized cells to form monoclonal antibody-producing hybridoma cells,
d) culturing the hybridoma cells, and
e) isolating from the culture monoclonal antibody which specifically binds to a polypeptide comprising an amino acid sequence selected from the group consisting of SEQ ID NO: 2 and SEQ ID NO: 4.

40. A monoclonal antibody produced by a method of claim 39.

41. A composition comprising the monoclonal antibody of claim 40 and a suitable carrier.

42. The antibody of claim 11, wherein the antibody is produced by screening a Fab expression library.

43. The antibody of claim 11, wherein the antibody is produced by screening a recombinant immunoglobulin library.

44. A method of detecting a polypeptide comprising an amino acid sequence selected from the group consisting of SEQ ID NO: 2 and SEQ ID NO: 4 in a sample, the method comprising:
a) incubating the antibody of claim 11 with a sample under conditions to allow specific binding of the antibody and the polypeptide, and
b) detecting specific binding, wherein specific binding indicates the presence of a polypeptide comprising an amino acid sequence selected from the group consisting of SEQ ID NO: 2 and SEQ ID NO: 4 in the sample.

45. A method of purifying a polypeptide comprising an amino acid sequence selected from the group consisting of SEQ ID NO: 2 and SEQ ID NO: 4 from a sample, the method comprising:
a) incubating the antibody of claim 11 with a sample under conditions to allow specific binding of the antibody and the polypeptide, and
b) separating the antibody from the sample and obtaining the purified polypeptide comprising an amino acid sequence selected from the group consisting of SEQ ID NO: 2 and SEQ ID NO: 4.

46. A microarray wherein at least one element of the microarray is a polynucleotide of claim 13.

47. A method of generating an expression profile of a sample which contains polynucleotides, the method comprising:
a) labeling the polynucleotides of the sample,
b) contacting the elements of the microarray of claim 46 with the labeled polynucleotides of the sample under conditions suitable for the formation of a hybridization complex, and
c) quantifying the expression of the polynucleotides in the sample.

48. An array comprising different nucleotide molecules affixed in distinct physical locations on a solid substrate, wherein at least one of said nucleotide molecules comprises a first oligonucleotide or polynucleotide sequence specifically hybridizable with at least 30 contiguous nucleotides of a target polynucleotide, and wherein said target polynucleotide is a polynucleotide of claim 12.

49. An array of claim 48, wherein said first oligonucleotide or polynucleotide sequence is completely complementary to at least 30 contiguous nucleotides of said target polynucleotide.

50. An array of claim 48, wherein said first oligonucleotide or polynucleotide sequence is completely complementary to at least 60 contiguous nucleotides of said target polynucleotide.

51. An array of claim 48, wherein said first oligonucleotide or polynucleotide sequence is completely complementary to said target polynucleotide.

52. An array of claim 48, which is a microarray.

53. An array of claim 48, further comprising said target polynucleotide hybridized to a nucleotide molecule comprising said first oligonucleotide or polynucleotide sequence.

54. An array of claim 48, wherein a linker joins at least one of said nucleotide molecules to said solid substrate.

55. An array of claim 48, wherein each distinct physical location on the substrate contains multiple nucleotide molecules, and the multiple nucleotide molecules at any single distinct physical location have the same sequence, and each distinct physical location on the substrate contains nucleotide molecules having a sequence which differs from the sequence of nucleotide molecules at another distinct physical location on the substrate.

56. A polypeptide of claim 1, comprising the amino acid sequence of SEQ ID NO: 2.

57. A polypeptide of claim 1, comprising the amino acid sequence of SEQ ID NO: 4.

58. A polynucleotide of claim 12, comprising the polynucleotide sequence of SEQ ID NO: 1.

59. A polynucleotide of claim 12, comprising the polynucleotide sequence of SEQ ID NO: 3.


SEQUENCE LISTING (6 sequences)

SEQ ID NO: 1 - DNA, 1557 bp, Homo sapiens
  misc_feature: Incyte ID No 478861
gaacaagatggattatcaagtgtcaagtccaatctatgacatcaattattatatatcggagccctgccaaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttcatacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagagtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaacgacacttttcatgtgtgaaaaaaaaaaaaaaaaa

SEQ ID NO: 2 - protein, 332 aa, Homo sapiens
  misc_feature: Incyte ID No 478861
MTSIIIYRSPAKKINVKQIAARLLPPLYSLVFIFGFVGNMLVILILINCKRLKSMTDIYLLNLAISDLFFLLTVPFWAHYAAAQWDFGNTMCQLLTGLYFIGFFSGIFFIILLTIDRYLAVVHAVFALKARTVTFGVVTSVITWVVAVFASLPGIIFTRSQKEGLHYTCSSHFHTLKIVILGLVLPLLVMVICYSGILKTLLRCRNEKKRHRAVRLIFTIMIVYFLFWAPYNIVLLLNTFQEFFGLNNCSSSNRLDQAMQVTETLGMTHCCINPIIYAFVGEKFRNYLLVFFQKHIAKRFCKCCSIFQQEAPERASSVYTRSTGEQEISVGL

SEQ ID NO: 3 - DNA, 1316 bp, Homo sapiens
  misc_feature: Incyte ID No 442279
tggggcgggtagagccaccaggggaatcaacagtggtttctcgtgcccctcagggtcaggagcagtctgatcaaaaggagggcatccactgtccggggccattcccacagctcccggatgctgggtctggaggctgcgcccttcccctgcaggagctcagcccagtgggcagtctgaagatggccaattacacgctggcaccagaggatgaatatgatgtcctcatagaaggtgaactggagagcgatgaggcagagcaatgtgacaagtatgacgcccaggcactctcagcccagctggtgccatcactctgctctgctgtgtttgtgatcggtgtcctggacaatctcctggttgtgcttatcctggtaaaatataaaggactcaaacgcgtggaaaatatctatcttctaaacttggcagtttctaacttgtgtttcttgcttaccctgcccttctgggctcatgctgggggcgatcccatgtgtaaaattctcattggactgtacttsctgggcctgtacagtgagacayttttcaattgccttctgactgtgcaaaggtacctagtgtttttgcacaagggcaactttttctcagccaggaggagggtgccctgtggcatcattacaagtgtcctggcatgggtaacagccattctggccactttgcctgaatacgtggtttataaacctcagatggaagaccagaaatacaagtgtgcatttagcagaactcccttcctgccagctgatgagacattctggaagcattttctgactttaaaaatgaacatttcggttcttgtcctccccctatttatttttacatttctctatgtgcaaatgagaaaaacactaaggttcagggagcagaggtatagccttttcaagcttgtttttgccataatggtagtcttccttctgatgtgggcgccctacaatattgcatttttcctgtccactttcaaagaacacttctccctgagtgactgcaagagcagctacaatctggacaaaagtgttcacatcactaaactcatcgccaccacccactgctgcatcaaccctctcctgtatgcgtttcttgatgggacatttagcaaatacctctgccgctgtttccatctgcgtagtaacaccccacttcaacccagggggcagtctgcacaaggcacatcgagggaagaacctgaccattccaccgaagtgtaaactagcatccaccaaatgcaagaagaataaacatggattttcatctttctgcattaaaaaaaaaaaaaaaaaaaaaaaaaaaaaaaaaaaaaaaaaaaaaaa

SEQ ID NO: 4 - protein, 344 aa, Homo sapiens
  misc_feature: Incyte ID No 442279
MANYTLAPEDEYDVLIEGELESDEAEQCDKYDAQALSAQLVPSLCSAVFVIGVLDNLLVVLILVKYKGLKRVENIYLLNLAVSNLCFLLTLPFWAHAGGDPMCKILIGLYXLGLYSETXFNCLLTVQRYLVFLHKGNFFSARRRVPCGIITSVLAWVTAILATLPEYVVYKPQMEDQKYKCAFSRTPFLPADETFWKHFLTLKMNISVLVLPLFIFTFLYVQMRKTLRFREQRYSLFKLVFAIMVVFLLMWAPYNIAFFLSTFKEHFSLSDCKSSYNLDKSVHITKLIATTHCCINPLLYAFLDGTFSKYLCRCFHLRSNTPLQPRGQSAQGTSREEPDHSTEV

SEQ ID NO: 5 - protein, 30 aa, Homo sapiens
  misc_feature: Incyte ID No UNKNOWN
IFFIILLTIDRYLAVVHAVFALKARTVFGV

SEQ ID NO: 6 - protein, 30 aa, Homo sapiens
  misc_feature: Incyte ID No UNKNOWN
IFFIILLTIDRYLAVVHAVFALRARTVFGV